(19) United States
(12) Patent Application Publication
(10) Pub. No.: US 2005/0049201 A1
(43) Pub. Date: Mar. 3, 2005

(54) Detection and removal of lead using lead-binding proteins

(76) Inventors: Adrian M. Lowe, Newton, MA (US); Gerard J. Colpas, Holden, MA (US); Maureen A. Hamilton, Littleton, MA (US); Michael J. Salcius, Oxford, MA (US); Mitchell C. Sanders, West Boylston, MA (US)

Correspondence Address: HAMILTON, BROOK, SMITH & REYNOLDS, P.C., 530 VIRGINIA ROAD, P.O. BOX 9133, CONCORD, MA, 01742-9133, US

(21) Appl. No.: 10/498,292

(22) PCT Filed: Dec. 11, 2002
(86) PCT No.: PCT/US02/39711

Related U.S. Application Data
(60) Provisional application No. 60/339,521, filed on Dec. 11, 2001.

(51) Int. Cl. A61K 38/08; C07K 7/06; G01N 33/558

(52) U.S. Cl. 514/17; 530/330; 436/514

(57) ABSTRACT

Thymosin beta-4 (TB4), Thymosin beta-9 (TB9), and acyl-CoA binding protein (ACBP) are two proteins that possess the ability to bind lead in human tissue. These two proteins have a high affinity lead-binding region with a dissociation constant (K_{d}) of about 10⁻⁹ M that allows for a strong interaction between the metal and the protein. These proteins and their analogs and lead-binding fragments can be used to detect and remove lead from various media.

### RELATED APPLICATION

This application claims the benefit of U.S. Provisional Application No. 60/339,521 filed Dec. 11, 2001. The entire teachings of the above application are incorporated herein by reference.

### BACKGROUND OF THE INVENTION

The toxic effects of lead probably constitute the oldest occupational disease in the world. Lead is now so widely distributed in air, food, and water that a completely lead-free environment would be difficult if not impossible to achieve. Today, hazardous working conditions have been improved, exposure of children to flaking lead-base paints and lead-containing gasoline emissions has been reduced, and adult exposure to illicit whiskey and battery casings is less frequent. However, important public health issues still remain concerning environmental pollutants carrying lead, and exposure of children to lead-based products.

Metallic lead is slowly but consistently absorbed into the body via all routes except through the skin (however, some organic compounds containing lead are well absorbed through the skin). Absorption of lead dust via the respiratory system is the most common cause of industrial lead poisoning. The intestinal tract is the primary route of entry in non-industrial exposure. The rate of lead absorption via the gastrointestinal (“GI”) tract varies with the nature of the lead compound. For children aged <6 years, the Centers for Disease Control has defined an elevated blood lead level (BLL) as >10 μg/dL, but evidence exists for subtle effects at lower levels.

Once absorbed into the bloodstream from the respiratory or GI tract, lead becomes bound to erythrocytes (“red blood cells”) and is subsequently distributed to soft tissue such as bone marrow, liver, kidney, and the testes. Its half-life in these tissues is about thirty days. Lead also crosses the placenta and poses a potential hazard to a fetus in utero. Most lead that has entered the body is eventually bound in the skeleton; the half-life of lead elimination from bone is greater than twenty years. Lead also becomes bound in hair and nails. Most of the absorbed lead is excreted via renal elimination. However, it can also be eliminated through sweat and breast milk.

The pathology of lead poisoning stems from the ability of lead to form complexes with many biological compounds in the body. Lead can inhibit the activity of enzymes in a variety of organ systems. For example, lead encephalopathy is an important acute disorder usually seen in children who have ingested lead-based paints. The mortality rate of lead encephalopathy is high, and for effective treatment, immediate chelation therapy is essential. Chelation therapy sequesters lead from the bloodstream, and thus accelerates its elimination from the body. There are other pathologies associated with lead ingestion including kidney damage, hypochromic microcytic anemia, decreased fertility in women, as well as other diseases. The detection and subsequent removal of lead in a person's environment can obviate the pathophysiological consequences articulated above due to lead exposure and ingestion.

Currently there exists a need for effective lead detection. Concomitant with this need are equally important effective lead removal methods. The present invention provides for both of these contemporary needs.

### SUMMARY OF THE INVENTION

The present invention pertains to proteins that bind lead in human tissue, and the use of such proteins for detecting and/or removing lead from a sample, for example, from a biological fluid, tissue, or bone, or from materials such as water, paint, and other materials in which lead removal is desirable. It has been discovered that thymosin beta-4 (TB4) and acyl-CoA binding protein (ACBP) are two major physiological proteins that possess the ability to bind lead. Each of these proteins has a high affinity lead-binding region that allows for a strong interaction between the metal and the protein.

These lead-binding proteins and their artificially constructed analogs (and/or derivatives), as well as any other lead-binding proteins can be employed to detect and remove lead contamination from solutions, surfaces, and vapors. Additionally, lead-binding proteins, including TB4 and ACBP, lead-binding fragments thereof, and their analogs or variants can be used to remove contaminating lead from physiological systems such as blood, tissue, and bone. Further, bioremediation of lead from a contaminated medium, such as water or paint, can be effectuated by use of these proteins and their fragments, analogs, or variants.

Accordingly, in one aspect, the invention features an apparatus (e.g., a device) for detecting and/or removing lead in a sample (or a test sample), comprising a matrix to which is affixed (or immobilized) one or more lead-binding proteins, wherein the one or more lead-binding proteins comprises a polypeptide selected from the group consisting of acyl-CoA binding protein, thymosin beta-4, and thymosin beta-9, or lead-binding fragments or variants thereof (wherein the fragments or variants have biological activity, that is, they specifically bind lead). In one embodiment, the one or more lead-binding proteins is detectably labeled. The label can be, for example, a fluorescent label. Alternatively, the solid support can comprise a matrix that contains the lead-binding protein(s).

In another aspect, the invention features a method for detecting the presence or absence of lead in a sample, comprising contacting the sample with one or more lead-binding proteins under conditions sufficient for lead in the sample to specifically bind to the lead-binding protein, wherein the one or more lead-binding proteins comprises a polypeptide selected from the group consisting of acyl-CoA binding protein, thymosin beta-4, and thymosin beta-9, or lead-binding fragments or variants thereof; and detecting the presence of lead in the sample. In one embodiment, the one or more lead-binding proteins or lead-binding fragments or variants thereof are labeled with a detectable label, and detection occurs by detecting an alteration of the detectable label. Presence of lead in a sample is indicated by alteration of the detectable label, and absence of lead in a sample is indicated by a lack of alteration of the detectable label.

In still another aspect, the invention features a method for detecting the presence or absence of lead in a sample, comprising contacting the sample with one or more lead-binding proteins under conditions sufficient for binding of lead to the lead-binding protein, wherein the one or more lead-binding proteins are attached to a solid substrate, and wherein the one or more lead-binding proteins comprises a polypeptide selected from the group consisting of acyl-CoA binding protein, thymosin beta-4, and thymosin beta-9, or lead-binding fragments or variants thereof; and detecting the presence of lead in the sample by detecting the detectable label. In one embodiment, detecting the presence of lead in the sample comprises contacting the lead-binding protein or lead-binding fragments or variants thereof with a detectable label that differentiates between (e.g., the label exhibits a change in signal intensity or signal wavelength emitted) lead-binding proteins or lead-binding fragments or variants thereof that are bound with lead, and those that are not; and wherein detection occurs by detecting an alteration of the detectable label. Absence of lead in a sample is indicated by no alteration in the detectable label. In another embodiment, the solid substrate comprises a matrix and the lead-binding proteins are affixed to or immobilized in the matrix.

In another aspect, the invention features a method for removing lead from a sample, comprising contacting the sample with one or more lead-binding proteins affixed to a matrix, wherein the one or more lead-binding proteins comprises a polypeptide selected from the group consisting of acyl-CoA binding protein, thymosin beta-4, and thymosin beta-9, or lead-binding fragments or variants thereof, thereby removing lead from the sample.

In still another aspect, the invention features a device for detecting the presence or absence of lead in a sample. The device comprises a solid support to which is attached a matrix. The matrix comprises a ligand which is diffusable through the matrix and detectable, wherein the ligand specifically binds to lead, and wherein the ligand is localized to a specific area of the matrix. One or more lead-binding proteins are also affixed to, or immobilized in, the matrix and localized to a specific area of the matrix different from the ligand. The one or more lead-binding proteins comprise a polypeptide selected from the group consisting of acyl-CoA binding protein, thymosin beta-4, and thymosin beta-9, or lead-binding fragments or variants thereof. In one embodiment, the ligand comprises a chromogen. The matrix can be ionic, hydrophobic, or covalent. Examples of matrices include nitrocellulose, PVDF, DEAB-cellulose, and glass filters. Examples of chromogens include sodium sulfide and sodium rhodizonic acid. The chromagen can be attached to the matrix through week ionic or hydrophobic interactions known to one skilled in the art. In another embodiment, the area of the matrix containing the ligand and the area of the matrix comprising one or more lead-binding proteins, or lead-binding fragments or variants thereof, are located at opposite ends of the device, separated by matrix that does not contain ligand or protein.

In yet another aspect, the invention features a method of detecting the presence of lead in a sample, comprising contacting the sample with the above-described device, wherein the sample contacts the area comprising the ligand under conditions suitable for the lead in the sample to bind to the ligand; maintaining the device under conditions wherein the lead bound ligand diffuses to the area comprising the lead-binding protein, wherein the lead bound ligand binds to the protein and is immunobilized. Thus the ligand accumulates at the site of the lead-binding protein, and is detected in the specific area comprising one or more lead-binding proteins affixed to or in the matrix. In one embodiment, the ligand comprises a chromogen. The chromogen can be, for example, a visible dye, where immobilization of the dye at the lead-binding area of the matrix indicates that lead has been detected in the sample.

In any of the above methods, the one or more lead-binding proteins comprises a polypeptide selected from the group consisting of acyl-CoA binding protein, thymosin beta-4, thymosin beta-9, lead-binding fragments thereof, and variants thereof. Alternatively, any lead-binding protein or lead-binding fragment or variant thereof can be used. In another embodiment, the one or more lead-binding proteins consists of a polypeptide selected from the group consisting of acyl-CoA binding protein, thymosin beta-4, thymosin beta-9, and lead-binding fragments thereof. In another embodiment, the thymosin beta-4 protein comprises or consists of the sequence of SEQ ID NO: 1. In still another embodiment, the acyl-CoA binding protein comprises or consists of the sequence of SEQ ID NO: 2. In another embodiment, the thymosin beta-9 protein comprises or consists of the sequence of

SEQ ID NO: 9
(ADKPDLGEINSFDKAKLKKTETQEKNTLPT KETIEQEKQAK)
or
SEQ ID NO: 10
(ETQEKNTLPTKETIE).

In still another aspect, the invention features a lead-binding polypeptide comprising, consisting essentially of, or consisting of the motif EX₁X₂E-linker-EX₃X₄E, wherein each of X₁, X₂, X₃, and X₄ are residues capable of forming an alpha helix, and wherein the linker contains a turn motif. In one embodiment, the lead-binding polypeptide consists of the sequence of SEQ ID NO: 10. In another embodiment the linker contains a proline residue. Such a polypeptide can be used in any of the lead detecting and/or lead removal methods described herein.

In one embodiment of the invention, synthetic genes are constructed for TB4 and/or ACBP proteins, and other lead-binding proteins described herein. These synthetic genes are constructed such that their respective protein products are efficiently over-expressed in bacteria, for example, in E. coli. In the case of TB4, the synthetic gene can be constructed from four separately synthesized single-stranded DNA molecules. These molecules are designed such that they hybridize by Watson-Crick base pairing into a single DNA molecule incorporating (1) a gene suitable for expressing TB4, for example, in E. coli. (e.g., Genlank Accession Number P01253) and (2) overhanging 5′ sticky ends compatible with NdeI and XhoI restriction sites. The synthetic DNA is then ligated into a prokaryotic expression vector such as pET24a (available from Novagen, Inc. Madison, Wis.), which has been previously digested using NdeI and XhoI restriction enzymes.

In the case of ACBP, a gene can be generated from cDNA made from mRNA of normal human keratinocyte (NHK) cells using the polymerase chain reaction, and two synthetic DNA oligonucleotides. Some of the codons can be altered in order to provide more efficient protein expression. The primers can be synthesized according to the human ACBP mRNA sequence, as recorded in the GenBank database (Accession Number M15887). The primer sequence can be modified slightly in order to alter the codon usage of the amplified gene to that preferred by E. coli (Nakamura, Y., Gojobori, T. and Ikemura, T. (2000) Nucl. Acids Res. 28, 292).

In another embodiment of the present invention, an assay using one or more synthetically produced lead-binding proteins is described for the detection of lead. In a particular aspect of this embodiment, one or more naturally-occurring or synthetically produced lead-binding proteins are labeled with a chemical compound that facilitates fluorescence emission (i.e., a detectable label). Examples of such compounds are described herein, and include any dye or dye combination whose fluorescence is sensitive to a conformational change in the protein, which affects the immediate environment of the dye. The nature and intensity of the fluorescence is acutely dependent upon the conformation of the protein to which the dye is bound. Any alteration in the structural conformation will result in an altered fluorescence. The conformational change within the protein is brought about by the binding of one or more lead molecules to the protein. If the labeled protein is admixed or otherwise placed in contact with a medium such as a solid, liquid, vapor, or gas that contains lead, then the lead will bind to the lead-binding protein causing a conformational change in the protein. This conformational change will be reflected by a spectral shift in a marker, such as a fluorescent signal. Such a detectable label differentiates between lead-binding proteins bound with lead and those that are unbound.

In another embodiment of the instant invention, a method for removing lead from a sample is described. As used herein, the term “sample” or “test sample” can mean any medium, such as a surface (e.g., a painted surface such as a window sill), liquid, gas or vapor. A lead-binding protein, such as Thymosin beta-4 and/or ACBP, lead-binding fragments thereof, or their respective analogs are admixed or otherwise placed in contact with a putative lead-containing medium. Based upon the affinity for lead by a lead-binding protein, lead that is contained within or on the medium will bind to the lead-binding protein and thus be effectively removed from the medium. In a particular aspect of this embodiment, the lead-binding protein is affixed (or immunobilized) to an insoluble solid matrix. The solid matrix can be ionic, hydrophobic, or covalent. Examples of matrices include nitrocellulose, PVDF, DEAE-cellulose, and glass filters. This device is placed in contact with (close approximation with) the medium. Lead from the medium will bind to the lead-binding protein affixed to the device. After a suitable period, the device is removed from the medium, thereby effectuating the removal of the contaminating lead from the medium.

One example of a lead-detection device of the present invention is a solid support (for example, a plastic slide with the lead-binding protein directly attached to it. Another example of a device is a solid support with a matrix layered or coated onto it, where the lead-binding protein immobilized in the matrix (see, for example, FIG. 10). Another example of a device is a material, for example, a liquid or gel that can be directly applied to a surface containing lead. The material contains or consists of a matrix in which lead-binding-proteins are immunobilized. After applying the material to the lead-containing surface, the material can be removed, for example, by wiping or mopping, thereby effectively removing lead from the sample.

In still another embodiment, the use of ACBP and TB4 for chelation therapy to remove lead and other heavy metals from soft tissues, the blood stream, and the skeletal system of human patients is disclosed. Chelation therapy may have profound effects oil the removal of heavy metals that have been attributed to a number of diseases (such as heart disease) in addition to lead poisoning in general. The chelation can be formulated in any suitable solution, for example, isotonic saline solution. The chelation therapy can be administered in any suitable method, for example, by intravenous administration.

In another embodiment of the present invention, an assay using one or more naturally-occurring or synthetically produced lead-binding proteins is described for the detection of lead. In a particular aspect of this embodiment, one or more lead-binding proteins are attached to a solid material, such as nitrocellulose. A solution containing lead, which could also include a bodily fluid such as blood or saliva, is placed onto the solid material and absorbed. After washing, a dye molecule (i.e., a detectable label, such as sodium rhodizonic acid or sodium sulfide) is added that will bind to any lead-containing protein present in the solid material and will change color when detecting the lead-protein complex. The dye will only concentrate in the presence of the lead-binding protein, and thus lead in a sample can be detected. Furthermore, the approximate concentration of the lead in the solution being tested may be determined by a semi-quantitative measurement of the size or density of the color produced upon binding of the dye.

A diagnostic device utilizing this assay is also described that will produce a visible sign if a sufficient concentration of lead is present in the sample being tested. This device comprises a solid support that contains a ligand (e.g., a chromogen, such as a dye) that binds specifically to lead. The ligand can be contained in a matrix. For example, the solid support can be glass, plastic, a synthetic solid material, or any material that forms a rigid platform suitable for containing or supporting a matrix. The solid support can be flat, for example, a slide, or it can be shaped to hold the matrix, for example, a microtiter well, tube, cassette, column, cuvette, or capillary, and have an opening that permits contact between the lead-containing sample or surface and the matrix. In one embodiment, if the solid support encloses the matrix (except for the part of the region of the matrix that comes into contact with the lead-containing sample) the solid support is made of material that is translucent or transparent, or contains a window that is translucent or transparent. The matrix can comprise a natural or synthetic polymer in a solid or gel-like form, for example, nitrocellulose, agarose, collagen, hyaluronic acid, dextran, alginate, polyacrylamide, polyacrylate, polybuterate, polyurethane, silicone, rubber, nylon, vinyl, a resin, polyethylene, PVC, or any material that would permit diffusion or movement of the ligand toward the lead-binding protein region within a reasonable amount of time. Such matrix materials are well known in the art. The ligand can itself be a detectable label, or the ligand can be detectably labeled. The ligand is diffusable through the matrix when unbound and when bound to lead, and is localized to a specific region of the matrix. The device also comprises one or more lead-binding proteins affixed to (immobilized in) the matrix, and localized to the matrix in an area different from the area containing the ligand. In one embodiment, the areas of the matrix containing the ligand and the area to which the lead-binding proteins are affixed are at opposite ends of the device, and are separated by matrix that does not contain lead-binding protein or ligand.

When a sample is applied to the device, any lead present in the sample will react with the ligand (e.g., a chromogenic dye) to form a product (e.g., a lead-ligand complex) that is detectable (for example, a highly colored product). However, because the lead-binding complex is still fairly diffuse within the matrix, it may not be visible. However, the complex can diffuse through the matrix (medium) and bind to the localized lead-binding protein, where the detectably labeled lead will immobilize and accumulate. This accumulation can be detected by detecting the detectable label. For example, if the ligand is a chromogen, the area of the matrix containing the lead-binding proteins will be visible as a brightly colored area.

The invention also features kits for removing and/or detecting lead in a sample. The kits comprise the devices described herein and one or more reagents for detecting lead in a sample and/or instructions for use of the device.

### BRIEF DESCRIPTION OF THE DRAWINGS

FIG. 1 is the human amino acid sequence of TB4 (SEQ ID NO: 1).

FIG. 2 is the human amino acid sequence of ACBP (SEQ ID NO: 2).

FIG. 3A shows a synthesized single-stranded DNA molecule used to construct a synthetic modified TB4 gene.

FIG. 3B shows a synthesized single-stranded DNA molecule used to construct a synthetic modified TB4 gene.

FIG. 3C shows a synthesized single-stranded DNA molecule used to construct a synthetic modified TB4 gene.

FIG. 3D shows a synthesized single-stranded DNA molecule used to construct a synthetic modified TB4 gene.

FIG. 4 illustrates the construction of a synthetic TB4 gene from a single-stranded DNA molecule.

FIG. 5A shows a synthesized single-stranded DNA primer used to construct a synthetic ACBP gene. Uppercase letters indicate coding sequence.

FIG. 5B shows a synthesized single-stranded DNA primer used to construct a synthetic ACBP gene. Uppercase letters indicate coding sequence.

FIG. 6A is a scanned image of an SDS-PAGE gel of purified ACBP protein.

FIG. 6B is a scanned image of an SDS-PAGE gel of purified TB4 protein.

FIG. 7 is a scanned image of an SDS-PAGE gel of purified and fluorescently labeled proteins ACBP (lane 1) and TB4 (lane 2).

FIG. 8 is a graph of the relative fluorescence intensity, measured at 538 nm, for ACBP-dansyl in the presence and absence of lead molecules. A solution of 20 μM ACBP-dansyl in 20 mM phosphate buffer (pH 7.2) with 200 mM NaCl was titrated by addition of 5 mM lead acetate in water, and measured for fluorescence after incubation for 1 hour. The reactions were carried out in a 96-well microtitre plate with 150 μl total volume at 20° C.

FIG. 9A shows a solid material assay using TB4-45W bound to nitrocellulose in the presence of magnesium acetate, and sodium sulfide as the chromogenic dye

FIG. 9B shows a solid material assay using TB4-45W bound to nitrocellulose in the presence of lead acetate, and sodium sulfide as the chromogenic dye.

FIG. 9C shows a solid material assay using TB4-45W bound to nitrocellulose in the presence of lead acetate, detected using sodium sulfide as the dye.

FIG. 9D shows a solid material assay using ACBP (2.5 mg/ml) bound to nitrocellulose in the presence of lead acetate, detected using sodium sulfide as the dye.

FIG. 9E shows a solid material assay using TB4-S31C-45W (1.0 mg/ml) bound to nitrocellulose in the presence of lead acetate, detected using sodium sulfide as the dye.

FIG. 9F shows a solid material assay using TB4-S31C-45W (15.0 mg/ml) bound to nitrocellulose in the presence of lead acetate, detected using sodium sulfide as the dye.

FIG. 9G shows a solid material assay using TB4-45W (5.5 mg/ml) bound to nitrocellulose in the presence of lead acetate (10.0 μM), detected using sodium rhodizonic acid as the dye.

FIG. 9H shows a solid material assay using TB4-45W (5.5 mg/ml) bound to nitrocellulose in the presence of lead acetate (1.0 μM), detected using sodium rhodizonic acid as the dye.

FIG. 9I shows a solid material assay using TB4-45W (5.5 mg/ml) bound to nitrocellulose in the presence of lead acetate (0.1 μM), detected using sodium rhodizonic acid as the dye.

FIG. 10 shows a design for a diagnostic device that produces a color change in the presence of lead.

FIG. 11 is a graph of equilibrium data for TB4-45W (0 μM, 15 μM, 30 μM, 45 μM, and 60 μM) with 5 μM of lead as assessed using stripping voltammetry.

### DETAILED DESCRIPTION OF THE INVENTION

The present invention is based in part on the discovery that some proteins bind lead in human tissue. It has been discovered that thymosin beta-4 (TB4) and acyl-CoA binding protein (ACBP) are two such proteins that possess this ability to bind lead in human tissue. These two proteins have a high affinity lead-binding region with a dissociation constant (Kd) of about 10⁻⁹ M that allows for a strong interaction (e.g., binding) between the metal and the protein. (See Quintanilla-Vega et al. (1995) Chem. Biol. Interact. 98(3):193-209, and Smith et al. (1998) Chem. Biol. Interact. 115(1):39-52, the entire teachings of which are incorporated herein by reference.)

Thymosin beta-4 (TB4) is a forty-three amino acid polypeptide having a predicted molecular weight of around 5 kDa. This protein, originally thought to be a thymic hormone, has been demonstrated to be a potent regulator of cellular actin cyto skeletal networks. Thymosin beta-4, in vivo, sequesters actin monomers, thereby maintaining a pool of unpolymerized actin subunits and regulating the polymerization of actin filaments. The tertiary protein structure of TB4 consists primarily of a random coil with two small alpha helical regions. (For further biochemical analysis of TB4, see, Sanders et al. (1992) Proc. Natl. Acad. Sci. 89:4678-4682, the entire teachings of which are incorporated herein by reference.)

Acyl-CoA binding protein (ACBP) is a ninety-seven amino acid polypeptide having a predicted molecular weight of around 9 kDa. This protein is primarily thought to be involved in fatty acid synthesis. The protein sequesters long chain fatty acids in a biological cell to regulate lipid metabolism and biosynthesis. The tertiary structure of ACBP consists of a tight bundle of four alpha helices. (For further biochemical analysis, see, Kragelund et al. (1999) Biochim Biophys Acta. 1441(2-3):150-61, the entire teachings of which are incorporated herein by reference.)

Fragments and sequence variants of lead-binding proteins described herein that have lead-binding biological activity can also be used in the present invention. Variants include a substantially homologous polypeptide encoded by the same genetic locus in an organism, i.e., an allelic valiant, as well as other variants. Variants also encompass polypeptides derived from other genetic loci in an organism, but having substantial homology to a polypeptide of SEQ ID NO: 1, SEQ ID NO: 2, SEQ ID NO: 9, or SEQ ID NO: 10. Variants also include polypeptides substantially homologous or identical to these polypeptides but derived from another organism, i.e., an ortholog. Variants also include polypeptides that are substantially homologous or identical to these polypeptides that are produced by chemical synthesis. Variants also include polypeptides that are substantially homologous or identical to these polypeptides that are produced by recombinant methods. Such variants will have lead-binding activity.

As used herein, two polypeptides (or regions of the polypeptides) are substantially homologous or identical when the amino acid sequences are at least about 60%, 65%, 70%, 75%, 80%, 85%, 90%, or 95% or more homologous or identical.

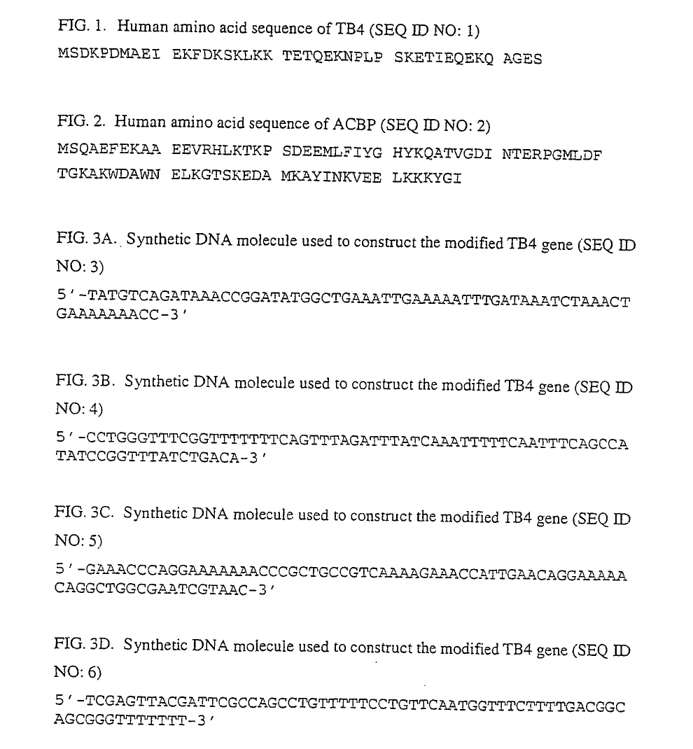

The percent identity of two nucleotide or amino acid sequences can be determined by aligning the sequences for optimal comparison purposes (e.g., gaps can be introduced in the sequence of a first sequence). The nucleotides or amino acids at corresponding positions are then compared, and the percent identity between the two sequences is a function of the number of identical positions shared by the sequences (i.e., % identity=#of identical positions/total # of positions×100). In certain embodiments, the length of the amino acid or nucleotide sequence aligned for comparison purposes is at least 30%, preferably, at least 40%, more preferably, at least 60%, and even more preferably, at least 70%, 80%, 90%, or 100% of the length of the reference sequence, for example, those sequences provided in FIGS. 1, 2, and 3, or SEQ ID NO: 9 or SEQ ID NO:10. The actual comparison of the two sequences can be accomplished by well-known methods, for example, using a mathematical algorithm. A preferred, non-limiting example of such a mathematical algorithm is described in Karlin et al. (1993) Proc. Natl. Acad. Sci. USA 90:5873-5877. Such an algorithm is incorporated into the BLASTN and BLASTX programs (version 2.2) as described in Schaffer et al. (2001) Nucleic Acids Res. 29:2994-3005. When utilizing BLAST and Gapped BLAST programs, the default parameters of the respective programs (e.g., BLASTN) can be used. In one embodiment, the database searched is a non-redundant (NR) database, and parameters for sequence comparison can be set at: no filters; Expect value of 10; Word Size of 3; the Matrix is BLOSUM62; and Gap Costs have an Existence of 11 and an Extension of 1.

Another preferred, non-limiting example of a mathematical algorithm utilized for the comparison of sequences is the algorithm of Myers and Miller, CABIOS (1989). Such an algorithm is incorporated into the ALIGN program (version 2.0), which is part of the GCG sequence alignment software package (Genetics Computer Group, Madison, Wis.). When utilizing the ALIGN program for comparing amino acid sequences, a PAM120 weight residue table, a gap length penalty of 12, and a gap penalty of 4 can be used. Additional algorithms for sequence analysis are known in the art and include ADVANCE and ADAM as described in Torellis and Robotti (1994) Comput. Appl. Biosci. 10: 3-5; and FASTA described in Pearson and Lipman (1988) Proc. Natl. Acad. Sci USA 85: 2444-8.

In another embodiment, the percent identity between two amino acid sequences can be accomplished using the GAP program in the GCG software package (Genetics Computer Group, Madison, Wis.) using either a Blossom 63 matrix or a PAM250 matrix, and a gap weight of 12, 10, 8, 6, or 4 and a length weight of 2, 3, or 4. In yet another embodiment, the percent identity between two nucleic acid sequences can be accomplished using the GAP program in the GCG software package, using a gap weight of 50 and a length weight of 3.

The invention also encompasses lead-binding polypeptides having a lower degree of identity but having sufficient similarity so as to perform one or more of the same functions, e.g., lead-binding activity, performed by a TB4 or ACBP polypeptide encoded by a nucleic acid molecule of the invention. Similarity is determined by conserved amino acid substitution. Such substitutions are those that substitute a given amino acid in a polypeptide by another amino acid of like characteristics. Conservative substitutions are likely to be phenotypically silent. Typically seen as conservative substitutions are the replacements, one for another, among the aliphatic amino acids Ala, Val, Leu, and De; interchange of the hydroxyl residues Ser and Thr; exchange of the acidic residues Asp and Glu; substitution between the amide residues Asn and Gln; exchange of the basic residues Lys and Arg; and replacements among the aromatic residues Phe and Tyr. Guidance concerning which amino acid changes are likely to be phenotypically silent are found in Bowie et al. (1990) Science 247: 1306-1310.

A variant polypeptide can differ in amino acid sequence by one or more substitutions, deletions, insertions, inversions, fusions, and truncations or a combination of any of these. Fully functional variants typically contain only conservative variation or variation in non-critical residues or in non-critical regions. Functional variants can also contain substitution of similar amino acids that result in no change or an insignificant change in function. Alternatively, such substitutions may positively or negatively affect function to some degree. Non-functional variants typically contain one or more non-conservative amino acid substitutions, deletions, insertions, inversions, or truncations or a substitution, insertion, inversion, or deletion in a critical residue or critical region, such critical regions include the lead-binding domain(s).

Amino acids that are essential for function (e.g., for lead-binding activity) can be identified by methods known in the art, such as site-directed mutagenesis or alanine-scanning mutagenesis (Cunningham et al. (1989) Science, 244: 1081-1085). The latter procedure introduces a single alanine mutation at each of the residues in the molecule (one mutation per molecule). The resulting mutant molecules are then tested for biological activity using methods described herein, or any or suitable method, for example, those described by Quintanilla-Vega et al. ((1995) Chem. Biol. Interact. 98(3):193-209), and Smith et al. ((1998) Chem. Biol. Interact. 115(1):39-52). Sites that are critical for polypeptide activity can also be determined by structural analysis, such as crystallization, nuclear magnetic resonance, or photoaffinity labeling (See Smith et al. (1992) J. Mol. Biol. 224: 899-904; and de Vos et al. (1992) Science 255: 306-312).

The invention also includes leading binding polypeptide fragments of the lead-binding proteins described herein of the invention. Fragments can be derived, for example, from a polypeptide comprising SEQ ID NO: 1, SEQ ID NO: 2, SEQ ID NO: 9, or SEQ ID NO: 10. The present invention also encompasses fragments of the variants of the polypeptides described herein. Useful fragments include those that retain lead-binding activity. Fragments of lead-binding proteins can be made using techniques known to one of skill in the art. The fragments can then be tested for lead-binding activity, using methods described herein, or any or suitable method, for example, those described by Quintanilla-Jega et al. ((1995) Chem. Biol. Interact. 98(3):193-209), and Smith et al. ((1998) Chem. Biol. Interact. 115(1):39-52). Fragments that have lead-binding activity can be used in the methods and devices of the present invention.

Biologically active fragments (peptides that are, for example, 6, 9, 12, 15, 16, 20, 30, 35, 40, 50, 60, 70, 80, 90, 95 or more amino acids in length) can comprise a domain, segment, or motif, for example, a lead-binding domain, that has been identified by analysis of the polypeptide sequence using well-known methods.

Fragments can be discrete (not fused to other amino acids or polypeptides) or can be within a larger polypeptide. Further, several fragments can be comprised within a single larger polypeptide. In one embodiment, a fragment designed for expression in a host can have heterologous pre- and pro-polypeptide regions fused to the amino terminus of the polypeptide fragment and an additional region fused to the carboxyl terminus of the fragment.

FIGS. 1 and 2 show the human amino acid sequences for TB4 and ACBP, respectively. Based upon these sequences, a synthetic gene comprising codons that facilitate efficient over-expression in bacteria was constructed independently for TB4 and ACBP.

The amino acid sequence of human TB4 was reverse translated into a degenerate DNA sequence. The codons were then substituted so as to retain the identical amino acid sequence, while using codons preferred by E. coli (Nakamura, Y., Gojobori, T. and Ikemura, T. (2000) Nucl. Acids Res. 28:292). Specifically, the TB4 gene was constructed from four separately synthesized single-stranded DNA molecules (SEQ ID NOs: 3-6) depicted in FIG. 3. The gene was constructed from four separate molecules to allow convenient automated synthesis. The single-stranded DNA molecules were designed such that they would hybridize by Watson-Crick base pairing into a single double-stranded DNA molecule. The four single stranded molecules were combined to form a single DNA as follows.

First, polypeptides consisting of SEQ ID NO: 3 and SEQ ID NO: 4 were mixed together in equimolar proportions, heated to 65° C. and slowly cooled to 40° C. over one hour. During this cooling process, the two molecules hybridized by Watson-Crick base pairing to form a single double stranded DNA that comprised the 5′ end of the TB4 gene.

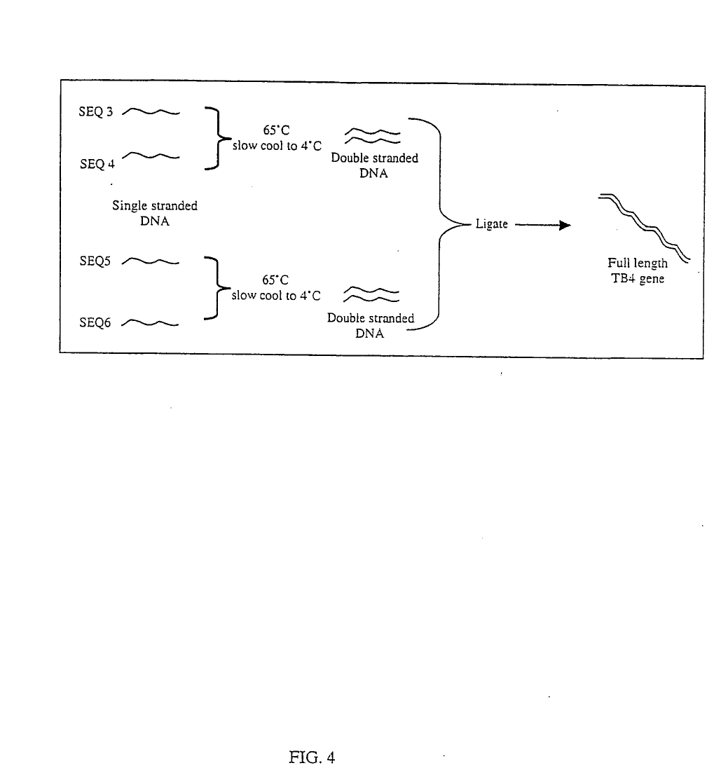

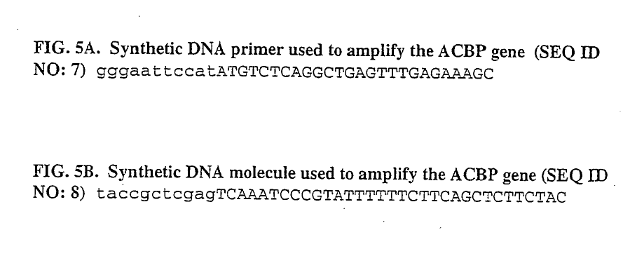

Next, polypeptides consisting of SEQ ID NO: 5 and SEQ ID NO: 6 were mixed together in equimolar proportions, heated to 65° C. and slowly cooled to 40° C. over one hour. During this cooling process, the two molecules hybridized by Watson-Crick base pairing to form a single double stranded DNA that comprised the 3′ end of the TB4 gene. Finally the two double stranded DNA molecules were ligated together by methods well known to those skilled in the art (Sambrook and Russell: Molecular Cloning, a laboratory manual 3rd Ed. 2001. Cold Spring Harbor Laboratory Press, NY). See FIG. 4.

The nascent DNA molecule was constructed so as to comprise a gene suitable for expressing TB4 in a host cell (e.g., bacterium such as E. coli). Moreover, the nascent DNA molecule was designed so as to comprise overhanging 5′ sticky ends compatible with NdeI and XhoI restriction sites. The nascent synthetic DNA molecule was subsequently ligated into a vector, such as pET24a (available from Novagen, Inc. Madison, Wis. by methods well known to those skilled in the art (Sambrook and Russell, Molecular Cloning, a laboratory manual 3rd Ed. 2001. Cold Spring Harbor Laboratory Press, NY), the teachings of which are incorporated herein in their entirety by reference). Prior to use, the vector was subjected to restriction using NdeI and XhoI. The bacterium, for example, E. coli, was then transformed with the new expression vector. Under suitable conditions well understood by those in the art, the recombinant TB4 gene expressed a protein product.

The ACBP gene was generated by PCR using two synthetic single stranded DNA oligonucleotide primers (SEQ ID NOs: 7 and 8, depicted in FIG. 5) that were designed to amplify the human ACBP gene, while altering some of the codons of the native gene to those more suitable for efficient expression in E. coli. The gene was amplified from cDNA generated from human NHK cell mRNA using methods well known to those skilled in the art (Sambrook and Russell., Molecular Cloning, a laboratory manual 3rd Ed. 2001. Cold Spring Harbor Laboratory Press, NY). In order to make the synthetic genes suitable for overexpression in E. coli, certain codons within the native gene sequences were altered. Specifically, codon 81 was changed from CTA to CTG, and codon 87 was changed from ATA to ATT by incorporating the altered codon sequences into the synthetic PCR primers. These changes allow for enhanced expression in E. coli, but did not alter the amino acid sequence of the ACBP protein. Under suitable conditions well understood by those in the art, the recombinant ACBP gene expressed a protein product.

Thymosin beta-9 (TB9), a human protein closely related to TB4, has been structurally characterized by NMR (Stoll et al. (1997) Biopolymers 41:623 and references contained therein). TB9 (SEQ ID NO: 9) has approximately 70% homology with TB4 and the structure of TB9 can serve as a model for the stricture of TB4. It is reasonable to believe that they share a similar function; therefore TB9 may also be capable of binding lead in the body and can serve the same function as TB4 in the examples described herein. Likewise, other proteins that are related to TB4 and ACBP may exist in the human genome or elsewhere and could be capable of performing similar functions iii vivo and in vitro. Examples of proteins (and their corresponding GenBank Accession Numbers) that have sequence homology to TB4 and ACBP are provided in Table 1 and Table 2. It is reasonable to expect that these proteins can also be, used bind lead, and to carry or the lead-binding and/or lead detection assays of the present invention.

TABLE 1
Proteins with Sequence Homology to TB4

| Accession | | |
|---|---|---|
| Number | Protein | Identity¹ |
| XM_111249 | Similar to Acyl-CoA binding protein | 5e−27 |
| XM_171679 | Similar to diazepam binding inhibitor | 9e−22 |
| BC029526 | Similar to Riken cDNA | 3e−21 |
| A60212 | Endogenous anti-morphine peptide | 6e−21 |
| AAAB01008846 | AgCP13099 | 7e−21 |
| AE003620 | CG8498 gene product | 2e−19 |
| NC_003423 | Probable acyl-coenzyme binding protein | 7e−18 |
| NM063929 | Enoyl CoA hydratase/isomerase | 3e−16 |
| NM021596 | Endozepine-like peptide | 7e−16 |
| NM_011868 | Peroxismal delta3, delta2, enoyl-CoA | 5e−15 |
| | isomerase | |
| BC001983 | Similar to peroxismal delta3, delta2, | 6e−15 |
| | enoyl-CoA isomerase | |
| AL136642 | Hypothetical protein | 1e−13 |
| NM_024722 | Hypothetical protein | 2e−13 |
| XM038526 | Similar to endozepine | 2e−10 |
| AE03568 | CG1704 gene product | 5e−10 |
| NM_124726 | Putative protein | 3e−07 |
| AY087475 | Putative Acyl-CoA binding protein | 3e−07 |
| NM_13998 | CG 5804 gene product | 1e−07 |
| AF229800 | Endozepine-like protein | 2e−06 |
| AE003560 | CG15829 gene product | 1e−06 |

¹Identity is expressed as the Expect Value, which is a parameter that measures the significance of match on a scale of 0 to 10; the closer the expect value is to 0, the more significant the match is.

TABLE 2
Proteins with Sequence Homology to ACBP

| Accession number | Protein | Identity¹ |
|---|---|---|
| XM_070564 | Similar to ribosomal protein L10 | 9e−11 |
| XM_093203 | Similar to CU240CZ | 7e−07 |
| XM_139460 | Similar to thymosin beta-10 | 1e−06 |
| AL133228 | DJ1071L10.1 novel thymosin/interferon | 2e−06 |
| | inducible multigene family | |
| XM_140953 | Similar to thymosin beta-10 | 4e−05 |
| XM_111908 | Similar to thymosin beta-4 | 1e−04 |
| AF452101 | Thymosin beta | 2e−04 |
| S22426 | Thymosin beta-12 rainbow trout | 2e−04 |
| P26352 | Thymosin beta-12 | 2e−04 |
| AJ25018 | Beta thymosin | 5e−04 |
| P26351 | Thymosin beta-11 | 5e−04 |
| S21282 | Thymosin beta-11 rainbow trout | 0.001 |
| P21753 | Thymosin beta-9 pig | 0.001 |
| XM_137746 | Similar to thymosin beta-4 | 0.001 |
| Q9I954 | Thymosin beta-b | 0.002 |
| NM_021103 | Thymosin beta-10 human | 0.002 |
| B19438 | Thymosin beta-9 | 0.002 |
| XM_151291 | Hypothetical protein | 0.20 |

¹Identity is expressed as the Expect Value, which is a parameter that measures the significance of match on a scale of 0 to 10; the closer the expect value is to 0, the more significant the match is.

It is reasonable to believe that the location of lead-binding to the ACBP and TB4 proteins is a site with sufficient carboxylic acid residues. The protein sequences include a number of aspartic acid and glutamic acid residues to serve as ligands to the lead ion. The TB4 protein binds lead in a 2:1 protein:lead ratio. The NMR structure of the bovine TB9 protein has been solved (Stoll, R et al. (1997) Biopolymers, 41:623), the entire teachings of which are incorporated herein by reference). The sequence of the bovine TB9 protein is 75% identical to the human TB4 protein. Several acidic residues are located on each helix near to the turn that connects them. The binding site proposed for TB4 includes the 3 glutamic acid residues located on the inside of a bend in the helical structure of the protein. When dimerized, these residues could provide 6 ligands to a lead ion and provide a binding pocket for the metal. The sequence from TB4 (human) that includes these residues and the connecting turn is a follows:

| | |
|---|---|
| 21-ETQEKNTLPTKETIE-35 | (SEQ ID NO: 10) |

Accordingly, it is reasonable to believe that the following motif can be used to bind lead: EX₁X₂E-linker-EX₃X₄E, wherein each of X₁, X₂, X₃, and X₄ are residues capable of forming an alpha helix, and wherein the linker contains a turn motif, for example, by comprising a proline residue. In one embodiment, the lead-binding motif comprises or consists of the sequence of SEQ ID NO: 10. In another embodiment, the leading-binding polypeptide comprises no more than 100 amino acids, no more than 90 amino acids, no more than 80 amino acids, no more than 70 amino acids, no more than 60 amino acids, no more than 50 amino acids, no more than 40 amino acids, no more than 30 amino acids, no more than 20 amino acids, or no more than 15 amino acids. In another embodiment, the polypeptide is not TB4 or TB9. Lead-binding variants and fragments of such polypeptides, including SEQ ID NO: 10 can also be used to carry out the lead-detection and lead-removal assays of the present invention. All of the glutamate residues in this region are conserved in both human and bovine TB4 and TB9 sequences. Polypeptides having the motif EX₁X₂E-linker-EX₃X₄E can be assessed for lead-binding activity using a method as described herein, or other lead-binding assays known to one skilled in the art.

The x-ray crystal structure of ACBP (bovine) has also been solved (Van Aalten, D. et al. (2001) J. Mol. Biol. 309:181). The difference between the bovine and human ACBP sequences is only a single amino acid. The structure of ACBP includes a number of acid residues that could potentially be used for binding lead. The number of glutamic acid and aspartic acid residues total 16 of 87 amino acids. There is no pocket containing 6 acid residues, the requisite number of ligands for a lead ion, however, one possible location includes the residues Glu 22, 23, 75, and 79. Another possible combination includes Glu 4, 75, and 78. A combination of these residues could be used in a monomeric binding mode, or possibly in a dimeric form as in TB4. Lead-binding activity of such polypeptides can be assessed using a method as described herein, or other lead-binding assays known to one skilled in the art.

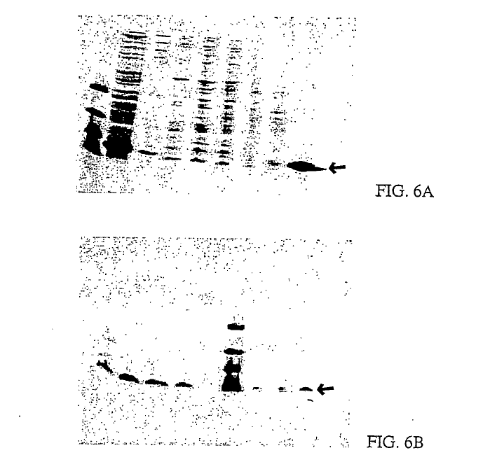

The recombinant TB4 and ACBP proteins, and other lead-binding proteins can be purified using techniques well known to those skilled in the art. One such technique involves acid extraction. Recombinant E. coli harboring the expression plasmid, which comprises the synthetic lead-binding protein, is grown at 37° C. in rich media, for example, Luria broth, in an erlenmeyer flask, with vigorous shaking to ensure adequate oxygenation of the culture. The culture is grown until the OD600 reaches about 1.0, and is then induced with isopropyl thiogalacto-pyranoside (IPTG), until maximal protein expression has occurred (approximately 90 minutes). The culture is then harvested by centrifugation (about 5000×g), and resuspended in 2M acetic acid, and homogenized on ice using, for example, a probe sonicator, or French press. Next, the preparation is centrifuged at about 12,000×g for approximately 20 minutes at about 4° C. This centrifugation process sediments the precipitated contaminating proteins in the preparation. The post-centrifugation supernatant contains the soluble recombinant lead-binding protein. Immediately following centrifugation, the supernatant is neutralized to a pH of about 7.0 using, for example, sodium hydroxide. This neutralization step is optimally monitored using techniques well known to those skilled in the art, such as a pH meter or pH indicator strip. The supernatant can be subjected to chromatography in order to further purify the recombinant protein. For example, reverse-phase, ion-exchange, size-exclusion chromatography or a combination thereof can be employed to further purify the desired protein. SDS-PAGE gels showing the recombinant ACBP and TB4 proteins in their various stages of purification are shown in FIGS. 6A and 6B, respectively. The proteins (indicated by the arrows) were purified by acid hydrolysis followed by quaternary amine ion exchange.

Once the purified recombinant protein has been secured, it can be conjugated with a marker, such as a fluorescence marker. Examples of markers include detectable labels. As used herein, a “detectable label” is a molecule(s) used for the detection of a substrate, and includes, for example, various enzymes, prosthetic groups, fluorescent materials, luminescent materials, bioluminescent materials, and radioactive materials. Examples of suitable enzymes include horseradish peroxidase, alkaline phosphatase, β-galactosidase, and acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, pyrene, dansyl (e.g., dansyl chloride) and phycoerythrin; an example of a luminescent material includes luminol; examples of bioluminescent materials include luciferase, luciferin, and aequorin, and examples of suitable radioactive material include ¹²⁵I, ¹³¹I, ³⁵S, ³H, and rhodamine 110. Other markers can be used that are well known in the art.

Pyrene and dansyl are two fluorescent dyes that exhibit marked changes in fluorescence intensity/wavelength when a protein substrate to which it is coupled undergoes a conformational change. Chemical coupling of N-(1-pyrene-butanoyl) cysteic acid, succinimidyl ester or dansyl aziridine to the native or recombinant TB4 and ACBP creates fluorescent analogs of these proteins, which are sensitive to the conformational changes that occur when they bind lead molecules. These fluorescent molecules can therefore be used as novel non-invasive sensors for detecting the presence of lead in a medium such as a gas, vapor, a surface, or liquid (including physiological fluids).

The lysine or methionine residues of native and recombinant TB4 and ACBP, and other lead-binding proteins, can be conjugated with N-(1-pyrenebutanoyl) cysteic acid, succinimidyl ester or dansyl aziridine, respectively, using methods well known to those skilled in the art. For example, the dye can be suspended in a small volume, for example, 50 μl of dimethyl sulfoxide or ethanol to a dye concentration of about 1 nm. This solution can then be added drop-wise to the purified protein solution while stirring on ice. The labeling reaction occurs during this mixing. The reaction time varies but generally proceeds for about one hour. The fluorescent conjugates can then be subsequently purified using; size-exclusion chromatography or any other suitable method known to one skilled in the art.

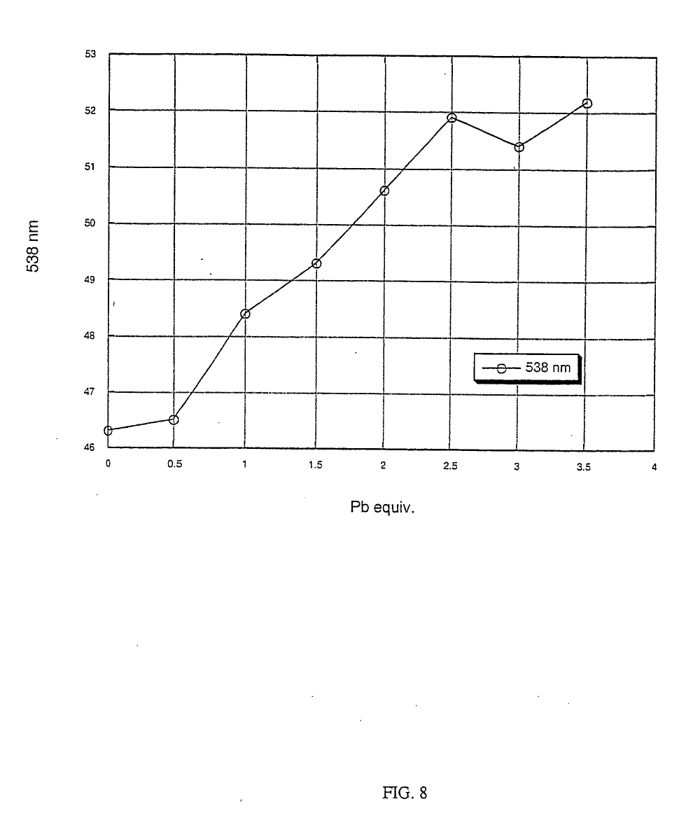

The fluorescent analogs of native and recombinant TB4 and ACBP, and other lead-binding proteins, are sensitive to intramolecular conformational changes, and can therefore be used as the first non-invasive sensors for detecting the presence of lead. In their respective lead-free states, the labeled proteins appear as a tight cluster of four alpha helices (ACBP) or a random coil (TB4). After exposure to, and binding of lead, the labeled proteins undergo a conformational change, and the dye becomes more fluorescently excited. The relative fluorescence increase caused by the addition of lead is shown in FIG. 8. Note the major spectral shift of the fluorescence signal in the presence of the lead in solution.

Alternatively, the ACBP and TB4 lead-binding proteins, and other lead-binding proteins can be labeled using iodoacetamide reactive dyes, which can be conjugated to cysteine residues. If no cysteine residues are available in a protein sequence, then single residue mutations can be introduced using methods well known to those skilled in the art, in order to provide cysteine residues for labeling. This is most conveniently achieved by changing a serine to a cysteine, as this is a very conservative change, and is unlikely to alter the function of the protein.

Purified TB4 and ACBP (including their respective analogs), and other lead-binding proteins can be immobilized on or in a matrix so that they can still bind lead from a medium such as a gas, vapor, air, liquid or a surface, while remaining affixed to or immobilized in an inert matrix, for example, cotton, glass microfibers, or polystyrene beads. The matrix can comprise a natural or synthetic polymer in a solid or gel-like form, for example, nitrocellulose, agarose, collagen, hyaluronic acid, dextran, alginate, polyacrylamide, polyacrylate, polybuterate, polyurethane, silicone, rubber, nylon, vinyl, a resin, polyethylene, PVC, or any material that would permit diffusion or movement of the ligand toward the region of the matrix to which the lead-binding protein is coupled within a reasonable amount of time. Such matrix materials are well known in the art. The leading binding proteins can be covalently coupled to the matrix, by means well known to those skilled in the art, or non-covalently bound by hydrophobic or ionic interactions of TB4 and/or ACBP, and other lead-binding proteins (free or labeled) with the matrix.

In addition, optionally, the matrix can be localized in or on a solid support. The solid substrate can be any solid support such as an inert plastic with a layer of matrix attached to the substrate. In another example, the solid support can be a container (e.g., a tube, a microtiter well, or a cassette) containing or coated (on the inside) with the matrix. Optionally, the container is transparent or translucent, or contains a transparent or translucent window through which binding of lead to the lead-binding protein can be detected. Lead-binding proteins or lead-binding fragments thereof can be coupled to matrix through ionic, covalent, or hydrophobic bonds. The proteins or fragments with hydrophobic leaving groups can be non-covalently bound to hydrophobic surfaces. Alternatively hydrophilic or hydrophobic lead-binding proteins or protein fragments can be coupled to surfaces by disulfide or primary amine, carboxyl or hydroxyl groups. Methods for coupling proteins to a solid substrate are known in the art. For example, proteins can be coupled to solid substrates using non-essential reactive termini such as free amines, carboxylic acids or thiol groups that do not effect the interaction with lead. Free amines can be coupled to carboxyl groups on the substrate using, for example, a 10 fold molar excess of either N-ethyl-N′-(3-dimethylaminopropyl)carbodiimide hydrochloride (EDC) or N-cyclohexyl-N′-2-(4′-methyl-morpholinium) ethyl carbodiimide-p-toluene sulphonate (CMC) for 2 hrs at 4° C. in distilled water adjusted to pH 4.5 to stimulate the condensation reaction to form a peptide linkage. Thiol groups can be reduced with DTT or TCEP and then coupled to a free amino group on a surface with N-e-Maleimidocaproic acid (EMCA, Griffith et al.(1981) FEBS Lett. 134:261-263). Another type of matrix to which the lead-binding protein or fragment thereof can be coupled to is QAE-sephadex, which binds TB4 at pH 7.5, and binds ACBP at pH 4.5.

Once the TB4 and/or ACBP, and other lead-binding proteins (including their respective analogs), labeled or free (unlabeled), are bound to or in the matrix, the matrix containing protein can be used independently or as part of a mechanical unit such as an air filter. These proteins are stable in environmental milieus, such as pH 4.0. The unlabeled protein can be employed to remove lead from the environment, whereas, the labeled protein can serve as a detection system for determining the presence of lead in a particular environment.

The TB4 and ACBP genes were further modified by site-directed mutagenesis to generate several synthetic variants (mutants, derivatives) of each. As the native form of TB4 does not have any significant absorption in the UV range, modification of the sequence to include a tryptophan residue would allow the protein to be readily observed by UV spectroscopy as an aid to purification. Modification of the DNA sequence by site-directed mutagenesis to add the codons for a tryptophan residue to the C-terminus of TB4 was performed to generate the mutant form called TB4-45W. As neither TB4 nor ACBP contain a cysteine, another modification made was the conservative replacement of a serine residue with a cysteine residue to generate a reactive site on the protein to be used to append a spectroscopic marker or for covalent attachment of the protein. Using the TB4-45W DNA sequence several more mutations were performed whereby all 4 of the serine residues in the TB4 sequence were individually changed to cysteine residues. Variants of TB4-45W generated include TB4-45W-S2C (in which serine at amino acid position 2 was changed to cysteine), TB4-45W-S16C (in which serine at amino acid position 16 was changed to cysteine), TB4-45W-S31C (in which serine at amino acid position 31 was changed to cysteine), and TB4-45W-S44C (in which serine at amino acid position 44 was changed to cysteine). Likewise the native sequence for ACBP was altered by site-directed mutagenesis to individually change all 3 serine residues to cysteine residues. Variants of ACBP include ACBP-S2C (in which serine at amino acid position 2 was changed to cysteine), ACBP-S21C (in which serine at amino acid position 21 was changed to cysteine), and ACBP-S66C (in which serine at amino acid position 66 was changed to cysteine). Under suitable conditions well understood by those skilled in the art, the recombinant TB4 and ACBP mutant genes expressed a protein product for purification.

A solid phase assay for the detection of lead was also developed using one or more synthetically produced lead-binding proteins. Both TB4 and ACBP and the various mutants that have been generated from them can be used in this assay. The experiment is performed by binding the protein to a solid surface support, such as nitrocellulose paper, cotton, glass microfibers, or polystyrene beads using, for example, methods described herein. Then the sample to be tested, such as saliva or blood, is placed onto the surface and allowed to react with the protein. After washing with buffer, or example a neutral non-chelating buffer to remove any unreacted material, the sample is treated with a ligand that can detect a lead-leading binding protein complex, for example, a ligand that can cause a color chance upon detecting a lead-protein complex. Examples of such ligands include, sodium sulfide and sodium rhodizonic acid. Furthermore, the concentration of the lead in the sample being tested may be determined by the intensity of the spot produced upon binding of the dye.

In an example of a typical assay 1.0 μl of the lead-binding protein (TB4, ACBP, or one of the various mutants) in an appropriate buffer solution was spotted onto a solid surface, such as nitrocellulose paper, and allowed to dry. A solution containing lead ions, such as 100 μM lead acetate in water, was placed over the solid material and allowed to incubate for approximately 30 seconds. The surface was washed several times with a buffer solution, such as Tris buffer (pH 7.5) with 200 mM NaCl. Then the surface was treated with a chromogenic dye molecule, such as 1.0 mM sodium sulfide in TBS (pH 8.5) or 1.0 mM sodium rhodizonic acid in 50 mM MES buffer (pH 5), and incubated for 1 to 5 minutes to allow the color to develop. The surface was then rinsed several times with water and allowed to dry.

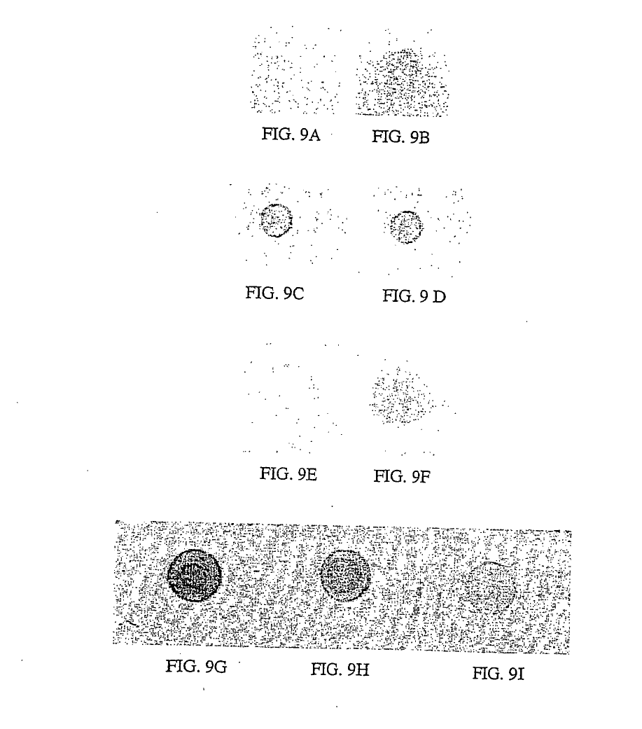

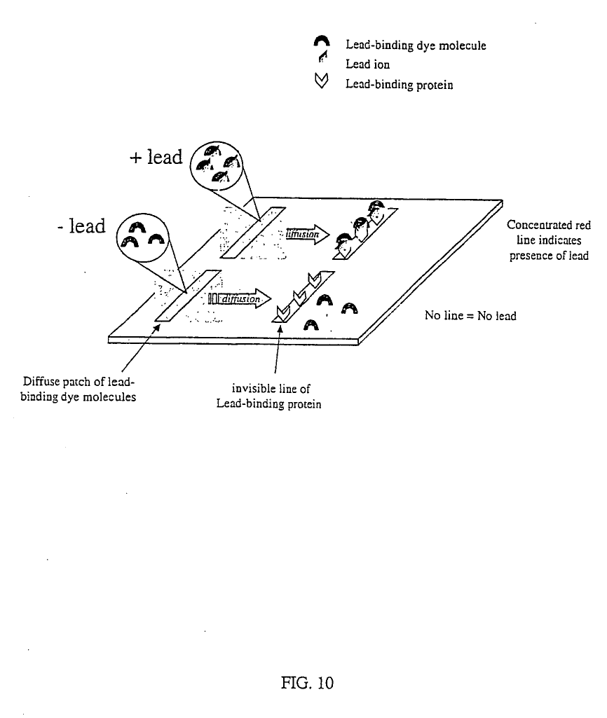

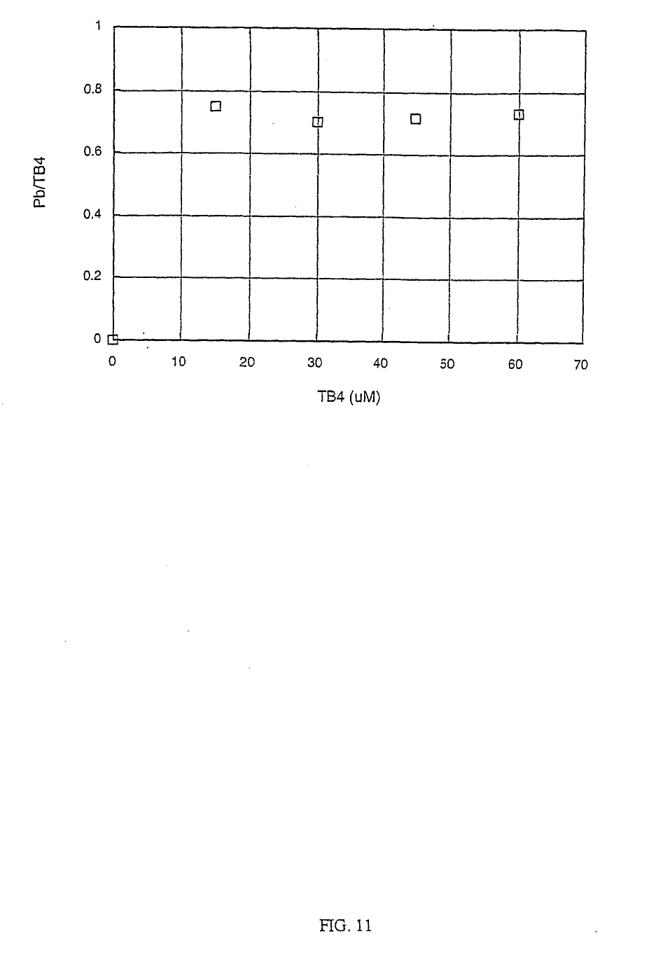

The results obtained for several individual examples of this assay are shown in FIGS. 9A-9I. As a control, the assay was tested using solutions containing several different buffers, pH conditions, and salts for reaction with the sodium sulfide and sodium rhodizonic acid dyes used. The samples labeled FIG. 9A and FIG. 9B show the reactions obtained with 100 μM magnesium acetate (FIG. 9A) and 100 μM lead acetate (FIG. 9B) using the conditions described above with TB4-45W as the lead-binding protein and sodium sulfide as the chromogenic dye. The assay can also be performed in the same manner with ACBP and the various mutants made from TB4 and ACBP. The samples labeled as FIGS. 9C and 9D show the results for ACBP (FIG. 9C) and TB4-45W (FIG. 9D) in the presence of 100 μM lead acetate, with sodium sulfide as the dye. The color change was dependent on the concentration of the bound protein as shown in the samples labeled as FIGS. 9E and 9F for low (1.0 mg/ml; FIG. 9E) and high (15.0 mg/ml; FIG. 9F) concentrations of TB4-S31C-45W with sodium sulfide. Likewise the color change was shown to be dependent on the concentration of lead. The samples labeled FIGS. 9G, 9H, and 9I show the color change observed for 10 μM (FIG. 9G), 1.0 μM (FIG. 9H), and 0.1 μM (FIG. 9I) lead acetate solutions used in an assay with TB4-45W and sodium rhodizonic acid. The pH was determined empirically for rhodizonic acid over a range of pH 4 to pH 8. Sodium sulfide was not stable in acid. The combined results of the experiments performed here indicate that the assay works well under various conditions with both TB4 and ACBP (and their variants, mutants, and derivatives).

To further demonstrate the detection and removal of lead using the proteins described herein, the following assay was carried out. The protein TB4-45W was prepared from E. coli cells using 100 mM acetic acid to extract the protein upon lysis. The extract was run on a QAB ion exchange column and eluted with a salt gradient. Fractions containing purified TB4-45W were collected and stored at −20 C. A 200 ml solution of 5 μM Pb(OAc)₂ in 5 mM Tris (pH=7.4) buffer was used to dialyze the protein in a beaker with stirring. The protein TB4-45W was placed in mini-dialysis cups and dialyzed overnight at 5° C. Aliquots were collected and analyzed for lead concentration by stripping voltammetry. The results are shown in Table 3.

TABLE 3
Stripping voltammetry data for lead in samples with TB4-45W.

| Sample | TB4 (μM) | Voltammetry¹ | Pb (μM)² |
|---|---|---|---|
| Buffer | 0 | 23 | (5) |
| TB4-1 | 15 | 103 | 11.2 |
| TB4-2 | 30 | 150 | 21 |
| TB4-3 | 45 | 192 | 28 |
| TB4-4 | 60 | 278 | 43.8 |

¹Stripping voltammetry data based on 100 ppb (0.24 μM) Pb standard = 58.
²Pb concentration calculated with buffer reading subtracted out.

Equilibrium binding data were then calculated for TB4-45W with the lead acetate (FIG. 11). The binding data indicates that the protein TB4-45W binds lead strongly in the micromolar range. The protein is reported to bind lead at a 2:1 (protein:metal) ratio in the nanomolar range. At micromolar concentrations, the 2:1 binding site plus some additional binding of lead by the protein was seen. It appears that a second binding mode is contributing at this high concentration. The concentration of lead used here is too high to measure the binding equilibrium; therefore the association constant must at least be in the nanomolar range.

An example of an application for this invention is a device or apparatus that produces a semi-quantitative measurement of the amount of lead in a test sample. As used herein, the terms “device” and “apparatus” are interchangeable. The device comprises a tube, for example, a plastic or glass tube containing one or more types of lead-binding proteins or fragments thereof, and a chromogenic ligand that binds lead, as described herein. A method for using such a device involves placing the lead-binding protein, or a lead-binding fragment thereof, in a narrow tube containing packed beads and a chromogenic ligand, for example, a chromogenic ligand as described herein. A lead containing sample is added to the top of the tube and allowed to diffuse into and through the tube. A color change occurs along the length of the tube as far as the lead travels. The length of the tube that changes color allows a determination of the amount of lead present in the sample.

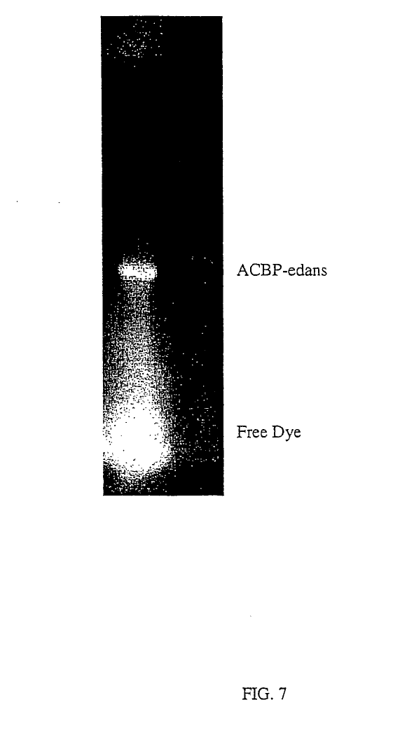

Another example of an application for this invention is water filtration. The matrix containing protein can be an integral part of a filter unit in a water purification system. Again, a dual role can be realized: first, detecting the presence of lead in the water; and second, the removal of lead from the water source can be effectuated. The relationship of bound versus free lead can be quantitated and subjected to analysis wherein first order kinetics can be observed along the linear section of a curve (FIG. 7). Further, this embodiment can include chemically coupling TB4 and/or ACBP, and other lead-binding proteins (including their respective analogs) to a reactive substrate or resin, such as CNBr Sepharose. The coupled substrate or resin can then be placed in a cartridge or disk-shaped filtration device for purposes of removing lead from a liquid such as water.

Still another application employing this matrix-bound protein is the removal of lead from a solid surface, using an absorbent material to which a lead-binding protein is affixed to or immobilized in. In one aspect of this embodiment, the matrix-bound protein is an integral part of an absorbent device, for example, a towel or a sponge-like device that is designed to remove lead from surfaces, such as those coated with a lead-based paint. The sponge-like protein-containing device can be applied to a surface under suitable conditions for removing contaminating lead from the surface. The matrix-bound protein component of the sponge can be integrated within the spongy component itself in a batch-wise manner. Alternatively, the matrix-bound protein can be integrated within the sponge in discrete zones. This can be achieved using methods known to one skilled in the art, for example, by crosslinking through disulfide bonds.

In still another embodiment, the invention features kits for removing and/or detecting lead in a sample. The kits comprise the devices described herein and one or more reagents for detecting lead in a sample and/or instructions for use of the device. The kit call contain, for example, control samples the contain lead or are lead-free, for use with the test sample. Such control sample provide quality control to the devices and methods described herein. In addition, the control sample may contain various concentrations of lead, thereby providing a means for quantitative analysis of lead concentrations in test samples. In methods that involve adding a detectable label, for example, a dye to the lead or lead-binding proteins after the two molecules have bound, the kit can comprise such a detectable label. The kit can further comprise devices for collecting the test sample and/or contacting the lead-binding proteins with the test sample (e.g., a dropper). Optionally associated with such kits can be a notice in the form prescribed by the manufacturer or a governmental agency regulating the manufacture, use or sale of the kit. The kit can be labeled with information regarding appropriate uses, steps involved in used of the kit, or the like.

In still another embodiment, TB4 and ACBP (including their variants, derivatives, and mutants) can be employed to chelate lead in a physiological system such as an animal, including humans. One or more lead-binding proteins can be introduced into a subject who has an abnormally high lead level. The proteins can be delivered in suitable pharmaceutical vehicles or carriers. Once the lead-binding proteins are in their proper position within the subject, for example, the circulatory system, they can interact with and bind to any lead present. The bound lead is now unavailable to interact with the subject's normal proteins.

The carrier and lead-binding protein composition can be sterile. The formulation should suit the mode of administration. Suitable pharmaceutically acceptable carriers include but are not limited to water, salt solutions (e.g., NaCl), saline, buffered saline, alcohols, glycerol, ethanol, gum arabic, vegetable oils, benzyl alcohols, polyethylene glycols, gelatin, carbohydrates such as lactose, amylose or starch, dextrose, magnesium stearate, talc, silicic acid, viscous paraffin, perfume oil, fatty acid esters, hydroxymethylcellulose, polyvinyl pyrolidone, etc., as well as combinations thereof. The pharmaceutical preparations can, if desired, be mixed with auxiliary agents, e.g., lubricants, preservatives, stabilizers, wetting agents, emulsifiers, salts for influencing osmotic pressure, buffers, coloring, flavoring and/or aromatic substances and the like that do not deleteriously react with the active compounds.

The composition, if desired, can also contain minor amounts of wetting or emulsifying agents, or pH buffering agents. The composition can be a liquid solution, suspension, emulsion, tablet, pill, capsule, sustained release formulation, or powder. The composition can be formulated as a suppository, with traditional binders and carriers such as triglycerides. Oral formulation can include standard carriers such as pharmaceutical grades of mannitol, lactose, starch, magnesium stearate, polyvinyl pyrollidone, sodium saccharine, cellulose, magnesium carbonate, etc.

Methods of introduction of these compositions include, but are not limited to, intradermal, intramuscular, intraperitoneal, intraocular, intravenous, subcutaneous, topical, oral and intranasal. Other suitable methods of introduction can also include rechargeable or biodegradable devices and slow or fast release polymeric devices. The pharmaceutical compositions of this invention can also be administered as part of a combinatorial therapy with other compounds, for example, other lead chelating agents.

The composition can be formulated in accordance with the routine procedures as a pharmaceutical composition adapted for administration to human beings. For example, compositions for intravenous administration typically are solutions in sterile isotonic aqueous buffer. Where necessary, the composition may also include a solubilizing agent and a local anesthetic to ease pain at the site of the injection. Generally, the ingredients are supplied either separately or mixed together in unit dosage form, for example, as a dry lyophilized powder or water free concentrate in a hermetically sealed container such as an ampule or sachette indicating the quantity of active compound. Where the composition is to be administered by infusion, it can be dispensed with an infusion bottle containing sterile pharmaceutical grade water, saline or dextrose/water. Where the composition is administered by injection, an ampule of sterile water for injection or saline can be provided so that the ingredients may be mixed prior to administration.

For topical application, nonsprayable forms, viscous to semi-solid or solid forms comprising a carrier compatible with topical application and having a dynamic viscosity preferably greater than water, can be employed. Suitable formulations include but are not limited to solutions, suspensions, emulsions, creams, ointments, powders, enemas, lotions, sols, liniments, salves, aerosols, etc., that are, if desired, sterilized or mixed with auxiliary agents, e.g., preservatives, stabilizers, wetting agents, buffers or salts for influencing osmotic pressure, etc. The compound may be incorporated into a cosmetic formulation. For topical application, also suitable are sprayable aerosol preparations wherein the active ingredient, preferably in combination with a solid or liquid inert carrier material, is packaged in a squeeze bottle or in admixture with a pressurized volatile, normally gaseous propellant, e.g., pressurized air.

Compounds described herein can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

The compounds are administered in a therapeutically effective amount. The amount of compounds that will be therapeutically effective in the treatment of a particular disorder or condition will depend on the nature of the disorder or condition, and can be determined by standard clinical techniques. In addition, in vitro or in vitro assays may optionally be employed to help identify optimal dosage ranges. The precise dose to be employed in the formulation will also depend on the route of administration, and the seriousness of the symptoms of a cell proliferation disease, an apoptotic disease, or a cell differentiation disease, and should be decided according to the judgment of a practitioner and each patient's circumstances. Effective doses may be extrapolated from dose-response curves derived from ill vitro or animal model test systems.

The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, that notice reflects approval by the agency of manufacture, use of sale for human administration. The pack or kit can be labeled with information regarding mode of administration, sequence of drug administration (e.g., separately, sequentially or concurrently), or the like. The pack or kit may also include means for reminding the patient to take the therapy. The pack or kit can be a single unit dosage of the combination therapy or it can be a plurality of unit dosages. In particular, the compounds can be separated, mixed together in any combination, present in a single vial or tablet. Compounds assembled in a blister pack or other dispensing means is preferred. For the purpose of this invention, unit dosage is intended to mean a dosage that is dependent on the individual pharmacodynamics of each compound and administered in FDA approved dosages in standard time courses.

The present invention also pertains to methods of treating a subject with an abnormally high level of lead using a lead-binding protein as described herein. Treating abnormally high levels of lead in a subject can be accomplished by delivering a lead-binding protein (e.g., TB4, ACBP, fragments thereof, or analogs thereof) to the subject. To deliver a therapeutic amount of a led binding protein to a subject in need thereof, it may be necessary to obtain large amounts of pure lead-binding protein from cultured cell systems, including bacterial cell culture systems which can express the protein. Delivery of the protein to the affected tissues can then be accomplished using appropriate packaging or administration systems.

The lead-binding polypeptides are administered in a therapeutically effective amount (i.e., an amount that is sufficient to treat the disease, such as by ameliorating symptoms associated with the disease, preventing or delaying the onset of the disease, and/or also lessening the severity or frequency of symptoms of the disease). The amount that will be therapeutically effective in the treatment of a particular individual's disorder or condition will depend on the symptoms and severity of the disease, and can be determined by standard clinical techniques. In addition, in vitro or in vivo assays may optionally be employed to help identify optimal dosage ranges. The precise dose to be employed in the formulation will also depend on the route of administration, and the seriousness of the disease or disorder, and should be decided according to the judgment of a practitioner and each patient's circumstances. Effective doses may be extrapolated from dose-response curves derived from in vitro or animal model test systems.

To facilitate the understanding of the present invention, a number of terms and phases are defined below:

As used herein, the terms “polynucleotide” and “oligonucleotide” are used interchangeably, and include polymeric forms of nucleotides of any length, either deoxyribonucleotides or ribonucleotides, or analogs thereof. Polynucleotides can have any three-dimensional structure, and can perform any function, known or unknown. The following are non-limiting examples of polynucleotides: a gene or gene fragment, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide can comprise modified nucleotides, such as methylated nucleotides and nucleotide analogs. If present, modifications to the nucleotide structure can be imparted before or after assembly of the polymer. The sequence of nucleotides may be interrupted by non-nucleotide components. A polynucleotide may be further modified after polymerization, such as by conjugation with a labeling component. The term also includes both double- and single-stranded molecules. Unless otherwise specified or required, any embodiment of this invention that is a polynucleotide encompasses both the double-stranded form and each of two complementary single-stranded forms known or predicted to make up the double-stranded form.

A polynucleotide is composed of a specific sequence of four nucleotide bases: adenine (A); cytosine (C); guanine (G); thymine (T); and uracil (U) for thymine when the polynucleotide is RNA. This, the term “polynucleotide sequence” is the alphabetical representation of a polynucleotide molecule. This alphabetical representation can be inputted into databases in a computer having a central processing unit and used for bioinformatics applications such as functional genomics and homology searching.

In one embodiment, the oligonucleotides or polynucleotides of the invention can include other appended groups such as peptides, e.g., for targeting host cell receptors in vivo, or agents facilitating transport across the cell membrane (see, e.g., Letsinger et al. (1989) Proc. Natl. Acad. Sci. USA 86:6553-6556; Lemaitre et al. (1987) Proc. Natl. Acad. Sci. USA 84:648-652; PCT Publication No. WO88/09810) or the blood-brain barrier (see, e.g., PCT Publication No. WO 89/10134). In addition, oligonucleotides can be modified with hybridization-triggered cleavage agents (see, Krol et al. (1988) Bio-Techniques 6:958-976) or intercalating agents (see, Zon (1988) Pharm. Res. 5:539-549). To this end, the oligonucleotide may be conjugated to another molecule, e.g., a peptide, hybridization triggered cross-linking agent, transport agent, or hybridization-triggered cleavage agent.

Finally, the oligonucleotide may be detectably labeled, either such that the label is detected by the addition of another reagent, e.g., a substrate for an enzymatic label, or is detectable immediately upon hybridization of the nucleotide, e.g., a radioactive label or a fluorescent label, e.g., a molecular beacon as described in U.S. Pat. No. 5,876,930.

As used herein, the term “nucleic acid molecule” is intended to include DNA molecules, e.g., cDNA or genomic DNA, and RNA molecules, e.g., mRNA, and analogs of the DNA or RNA generated using nucleotide analogs. The nucleic acid molecule can be single-stranded or double-stranded, but preferably is double-stranded DNA.

A nucleic acid molecule of the present invention, e.g., a nucleotide sequence encoding SEQ ID NOs: 1, 2, 9, or 10 or a portion thereof or having the lead-binding motif EX₁X₂E-linker-EX₃X₄E as described herein, can be isolated using standard molecular biology techniques and the sequence information provided herein. Using all or portion of the nucleic acid sequence of SEQ ED NOs: 3-8 or a nucleotide sequence encoding SEQ ID NOs: 1, 2, 3, 9, or 10 or the lead-binding motif EX₁X₂E-linker-EX₃X₄E as described herein as a hybridization probe, a molecule comprising SEQ ID NOs: 3-8, or a nucleotide sequence encoding SEQ ID NOs: 1, 2, 3, 9, or 10 or the lead-binding motif EX₁X₂E-linker-EX₃X₄E cal be isolated using standard hybridization and cloning techniques as described in Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989.

A nucleic acid of the invention can be amplified using cDNA, in RNA or alternatively, genomic DNA, as a template and appropriate oligonucleotide primers according to standard PCR amplification techniques. The nucleic acid so amplified can be cloned into an appropriate vector and characterized by DNA sequence analysis. Furthermore, oligonucleotides corresponding to marker nucleotide sequences, or nucleotide sequences encoding a marker of the invention can be prepared by standard synthetic techniques, e.g., using an automated DNA synthesizer.

In another embodiment, a nucleic acid molecule of the invention comprises a nucleic acid molecule which is a complement of the nucleotide sequence of SEQ ID NOs: 3-8, or a nucleotide sequence encoding SEQ ID NOs: 1, 2, 9, or 10 or a portion thereof, for example, a lead-binding portion thereof, or is a complement of a nucleotide sequence encoding the lead-binding motif EX₁X₂E-linker-EX₃X₄E. A nucleic acid molecule that is complementary to such a nucleotide sequence is one which is sufficiently complementary to the nucleotide sequence such that it can hybridize to the nucleotide sequence, thereby forming a stable duplex.

The nucleic acid molecule of the invention, moreover, can comprise only a portion of the nucleic acid sequence of SEQ ID NOs: 3-8 or a nucleotide sequence encoding SEQ ID NOs: 1, 2, 9, or 10 or encoding or the lead-binding motif EX₁X₂E-linker-EX₃X₄E of the invention, or a fragment thereof which can be used as a probe or primer. The probe/primer typically comprises substantially purified oligonucleotide.

Probes based on the nucleotide sequence of a nucleic acid molecule comprising SEQ ID NOs: 3-8 or a nucleotide sequence encoding SEQ ID NOs: 1, 2, 9, or 10 or encoding the lead-binding motif EX₁X₂E-linker-EX₃X₄E can be used to detect transcripts or genomic sequences corresponding to TB4, ACBP, TB9 or other lead-binding proteins as described herein. In other embodiments, the probe comprises a labeling group attached thereto, e.g., the labeling group can be a radioisotope, a fluorescent compound, an enzyme, or an enzyme co-factor. Such probes can be used as a part of a diagnostic test kit for identifying cells or tissue which misexpresses, e.g., over- or under-express, a polypeptide of the invention, or which have greater or fewer copies of a gene of the invention.

As used herein, the term “hybridizes under stringent conditions” is intended to describe conditions for hybridization and washing under which nucleotide sequences at least 60% homologous to each other typically remain hybridized to each other. Preferably, the conditions are such that sequences at least about 70%, more preferably at least about 80%, even more preferably at least about 85% or 90% homologous to each other typically remain hybridized to each other. Such stringent conditions are known to those skilled in the art and can be found in Current Protocols in Molecular Biology, John Wiley & Sons, N.Y. (1989), 6.3.1-6.3.6. A preferred, non-limiting example of stringent hybridization conditions are hybridization in 6× sodium chloride/sodium citrate (SSC) at about 45° C., followed by one or more washes in 0.2×SSC, 0.1% SDS at 50° C., preferably at 55° C., more preferably at 60° C., and even more preferably at 65° C. Preferably, an isolated nucleic acid molecule of the invention that hybridizes under stringent conditions to the sequence of SEQ ID NOs: 3-8 or a nucleotide sequence encoding SEQ ID NOs: 1, 2, 9, or 10 or encoding or the lead-binding motif EX₁X₂E-linker-EX₃X₄E. As used herein, a “naturally-occuring” nucleic acid molecule includes an RNA or DNA molecule having a nucleotide sequence that occurs in nature, e.g., encodes a natural protein.

A “gene” includes a polynucleotide containing at least one open reading frame that is capable of encoding a particular polypeptide or protein after being transcribed and translated. Any of the polynucleotide sequences described herein may be used to identify larger fragments or full-length coding sequences of the gene with which they are associated. Methods of isolating larger fragment sequences are known to those of skill in the art, some of which are described herein.

A “primer” includes a short polynucleotide, generally with a free 3′-OH group that binds to a target or “template” present in a sample of interest by hybridizing with the target, and thereafter promoting polymerization of a polynucleotide complementary to the target. A “polymerase chain reaction” (“PCR”) is a reaction in which replicate copies are made of a target polynucleotide using a “pair of primers” or “set of primers” consisting of “upstream” and a “downstream” primer, and a catalyst of polymerization, such as a DNA polymerase, typically a thermally-stable polymerase enzyme. Methods for PCR are well known in the art, and are taught, for example, in MacPherson et al., IRL Press at Oxford University Press (1991). All processes of producing replicate copies of a polynucleotide, such as PCR or gene cloning, are collectively referred to herein as “replication”. A primer can also be used as a probe in hybridization reactions, such as Southern or Northern blot analyses (see, for example, Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989).

The term “cDNAs” includes complementary DNA, that is mRNA molecules present in a cell or organism made into cDNA with an enzyme such as reverse transcriptase. A “cDNA library” includes a collection of mRNA molecules present in a cell or organism, converted into cDNA molecules with the enzyme reverse transcriptase, then inserted into “vectors” (other DNA molecules that can continue to replicate after addition of foreign DNA). Exemplary vectors for libraries include bacteriophage, viruses that infect bacteria, e.g., 1 phage. The library can then be probed for the specific cDNA (and thus mRNA) of interest.

The term “polypeptide” includes a compound of two or more subunit amino acids, amino acid analogs, or peptidomnimetics. The subunits may be linked by peptide bonds. In another embodiment, the subunit may be linked by other bonds, e.g., ester, ether, etc. As used herein the tern “amino acid” includes either natural and/or unnatural or synthetic amino acids, including glycine and both the D or L optical isomers, and amino acid analogs and peptidomimetics. A peptide of three or more amino acids is commonly referred to as an oligopeptide. Peptide chains of greater than three or more amino acids are referred to as a polypeptide or a protein.

A “host cell” is intended to include any individual cell or cell culture that can be or has been a recipient for vectors or for the incorporation of exogenous nucleic acid molecules, polynucleotides and/or proteins. It also is intended to include progeny of a single cell. The progeny may not necessarily be completely identical (in morphology or in genomic or total DNA complement) to the original parent cell due to natural, accidental, or deliberate mutation. The cells may be prokaryotic, and include but are not limited to bacterial cells. As used herein, “expression” includes the process by which polynucleotides are transcribed into mRNA and translated into peptides, polypeptides, or proteins. If the polynucleotide is derived from genomic DNA, expression may include splicing of the mRNA, if an appropriate eukaryotic host is selected. Regulatory elements required for expression include promoter sequences to bind RNA polymerase and transcription initiation sequences for ribosome binding. For example, a bacterial expression vector includes a promoter such as the lac promoter and for transcription initiation the Shine-Dalgarno sequence and the start codon AUG (Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, NA, 1989). Similarly, a eukaryotic expression vector includes a heterologous or homologous promoter for RNA polymerase II, a downstream polyadenylation signal, the start codon AUG, and a termination co don for detachment of the ribosome. Such vectors can be obtained commercially or assembled by the sequences described in methods well known in the art, for example, the methods described below for constructing vectors in general.

“Hybridization” includes a reaction in which one or more polynucleotides react to form a complex that is stabilized via hydrogen bonding between the bases of the nucleotide residues. The hydrogen bonding may occur by Watson-Crick base pairing, Hoogstein forming a duplex structure, three or more strands forming a multi-stranded complex, a single self-hybridizing strand, or any combination of these. A hybridization reaction may constitute a step in a more extensive process, such as the initiation of a PCR reaction, or the enzymatic cleavage of a polynucleotide by a ribozyme.

Hybridization reactions can be performed under conditions of different “stringency.” The stringency of a hybridization reaction includes the difficulty with which any two nucleic acid molecules will hybridize to one another. Under stringent conditions, nucleic acid molecules at least 60%, 65%, 70%, 75% identical to each other remain hybridized to each other, whereas molecules with low percent identity cannot remain hybridized. A preferred, non-limiting example of highly stringent hybridization conditions are hybridization in 6× sodium chloride/sodium citrate (SSC) at about 45° C., followed by one or more washes in 0.2 SSC, 0.1% SDS at 50° C., preferably at 55° C., more preferably at 60° C., and even more preferably at 65° C.

When hybridization occurs in an antiparallel configuration between two single-stranded polynucleotides, the reaction is called “annealing” and those polynucleotides are described as “complementary.” A double-stranded polynucleotide can be “complementary” or “homologous” to another polynucleotide, if hybridization can occur between one of the strands of the first polynucleotide and the second. “Complementary” or “homology” (the degree that one polynucleotide is complementary with another) is quantifiable in terms of the proportion of bases in opposing strands that are expected to hydrogen bond with each other, according to generally accepted base-pairing rules.

As used herein, by “test sample” is meant a sample that is examined for information, for example, using the methods described herein. The test sample can be examined for the presence or absence of lead. The test sample can be a biological sample, for example, a tissue biopsy, bone biopsy, cells, blood, serum, stool obtained from a patient or test subject. The test sample can also be an environmental sample, for example, air, dust, water supplies, or soil. In addition, the test sample can be a household item., for example, paint (in the can) or on a wall, dust, and tap water.

Another aspect of the invention pertains to vectors, preferably expression vectors, containing a nucleic acid encoding a marker protein of the invention (or a portion thereof). As used herein, the term “vector” includes a nucleic acid molecule capable of transporting another nucleic acid to which it has been linked. One type of vector is a “plasmid,” which includes a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced, e.g., bacterial vectors having a bacterial origin of replication and episomal mammalian vectors. Other vectors, e.g., non-episomal mammalian vectors, are integrated into the genome of a host cell upon introduction into the host cell, and thereby are replicated along with the host genome. Moreover, certain vectors are capable of directing the expression of genes to which they are operatively linked. Such vectors are referred to herein as “expression vectors.” In general, expression vectors of utility in recombinant DNA techniques are often in the form of plasmids. In the present specification, “plasmid” and “vector” can be used interchangeably as the plasmid is the most commonly used form of vector.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, which is operatively linked to the nucleic acid sequence to be expressed. Within a recombinant expression vector, “operatively linked” is intended to mean that the nucleotide sequence of interest is linked to the regulatory sequence(s) in a manner which allows for expression of the nucleotide sequence, e.g., in an in vitro transcription/translation system or in a host cell when the vector is introduced into the host cell. The term “regulatory sequence” is intended to include promoters, enhancers and other expression control elements, e.g., polyadenylation signals. Such regulatory sequences are described, for example, in Goeddel; Gene Expression Technology: Methods in Enzymology 185, Academic Press, San Diego, Calif. (1990). Regulatory sequences include those which direct constitutive expression of a nucleotide sequence in many types of host cells and those which direct expression of the nucleotide sequence only in certain host cells, e.g., tissue-specific regulatory sequences. It will be appreciated by those skilled in the art that the design of the expression vector can depend on such factors as the choice of the host cell to be transformed, the level of expression of protein desired, and the like. The expression vectors of the invention can be introduced into host cells to thereby produce proteins or peptides, including fusion proteins or peptides, encoded by nucleic acids as described herein, e.g., marker proteins, mutant forms of marker proteins, fusion proteins, and the like.

The recombinant expression vectors of the invention can be designed for expression of marker proteins in prokaryotic or eukaryotic cells. For example, proteins can be expressed in bacterial cells such as E. coli, insect cells (using baculovirus expression vectors) yeast cells or mammalian cells. Suitable host cells are discussed further in Goeddel, Gene Expression Technology: Methods in Enzymology 185, Academic Press, San Diego, Calif. (1990). Alternatively, the recombinant expression vector can be transcribed and translated in vitro, for example, using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith, D. B. and Johnson, K. S. (1988) Gene 67:31-40), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein.

Purified fusion proteins can be utilized in marker activity assays, e.g., direct assays or competitive assays described in detail below, or to generate antibodies specific for marker proteins, for example.

Examples of suitable inducible non-fusion E. coli expression vectors include pTrc (Amann et al. (1988) Gene 69:301-315) and pET 11d (Studier et al., Gene Expression Technology: Methods in Enzymology 185, Academic Press, San Diego, Calif. (1990) 60-89). Target gene expression from the pTrc vector relies on host RNA polymerase transcription from a hybrid trp-lac fusion promoter. Target gene expression from the pET 11d vector relies on transcription from a T7 gn10-lac fusion promoter mediated by a coexpressed viral RNA polymerase (T7 gn1). This viral polymerase is supplied by host strains BL21 (DE3) or HMS 174(DE3) from a resident prophage harboring a T7 gn1 gene under the transcriptional control of the lacUV 5 promoter.

One strategy to maximize recombinant protein expression in E. coli is to express the protein in a host bacteria with an impaired capacity to proteolytically cleave the recombinant protein (Gottesman, S., Gene Expression Technology: Methods in Enzymology 185, Academic Press, San Diego, Calif. (1990) 119-128). Another strategy is to alter the nucleic acid sequence of the nucleic acid to be inserted into an expression vector so that the individual codons for each amino acid are those preferentially utilized in E. coli (Wada et al. (1992) Nucleic Acids Res. 20:2111-2118). Such alteration of nucleic acid sequences of the invention can be carried out by standard DNA synthesis techniques.

Another aspect of the invention pertains to host cells into which a nucleic acid molecule of the invention is introduced within a recombinant expression vector or a nucleic acid molecule of the invention containing sequences which allow it to homologously recombine into a specific site of the host cell's genome. The terms “host cell” and “recombinant host cell” are used interchangeably herein. It is understood that such tenrs refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. Preferably, the host cell is a prokaryotic cell. For example, the invention can be expressed in bacterial cells such as E. coli. Other suitable host cells are known to those skilled in the art.

Vector DNA can be introduced into host cells via conventional transformation or transfection techniques. As used herein, the terms “transformation” and “transfection” are intended to refer to a variety of art-recognized techniques for introducing foreign nucleic acid, e.g., DNA, into a host cell, including calcium phosphate or calcium chloride co-precipitation, DEAE-dextran-mediated transfection, lipofection, or electroporation. Suitable methods for transforming or transfecting host cells can be found in Sambrook, et al. (Molecular Cloning: A Laboratory Manual. 2nd, ed., Cold Spring Harbor Laboratory, Cold Spring Harbor Laboratory Press, Cold Spring Harbor, N.Y., 1989), and other laboratory manuals.

A host cell of the invention, such as a host cell in culture, can be used to produce, i.e., express, a recombinant protein. Accordingly, the invention further provides methods for producing a protein using the host cells of the invention. In one embodiment the method comprises culturing the host cell of invention (into which a recombinant expression vector encoding a protein, or proteins, has been introduced) in a suitable medium such that a protein of the invention is produced. In another embodiment, the method further comprises isolating a protein from the medium or the host cell.

Of course, one skilled in the art will appreciate further features and advantages of the invention based on the above-described embodiments. Accordingly, the invention is not to be limited by what has been particularly shown and described, except as indicated by the appended claims.

While this invention has been particularly shown and described with references to preferred embodiments thereof, it will be understood by those skilled in the art that various changes in form and details may be made therein without departing from the scope of the invention encompassed by the appended claims.

1. A lead-binding polypeptide comprising the motif EX₁X₂E-liner-EX₃X₄E, wherein each of X₁, X₂, X₃, and X₄ are residues capable of forming an alpha helix, and wherein the linker contains a turn motif.

2. The lead-binding polypeptide of claim 1, wherein said polypeptide consists of the sequence of SEQ ID NO: 10.

3. A device for detecting and/or removing lead in a sample, comprising a matrix to which is affixed one or more lead-binding proteins, wherein said one or more lead-binding proteins comprises a polypeptide having the motif EX₁X₂E-linker-EX₃X₄E, wherein each of X₁, X₂, X₃, and X₄ are residues capable of forming an alpha helix, and wherein the linker contains a turn motif.

4. A device for detecting and/or removing lead in a sample, comprising a matrix to which is affixed one or more lead-binding proteins, wherein said one or more lead-binding proteins comprises a polypeptide selected from the group consisting of acyl-CoA binding protein, thymosin beta-4, and thymosin beta-9, or lead-binding fragments or variants thereof.

5. The device of claim 4, wherein said thymosin beta-4 protein comprises the sequence of SEQ ID NO: 1 or SEQ ID NO: 10.

6. The device of claim 4, wherein said acyl-CoA binding protein comprises the sequence of SEQ ID NO: 2.

7. The device of claim 4, wherein said thymosin beta-9 protein comprises the sequence of SEQ ID NO: 9.

8. The device of claim 4, wherein said one or more lead-binding proteins is detectably labeled.

9. The device of claim 8, wherein said label comprises a fluorescent label.

10. A method for detecting the presence of lead in a sample, comprising:
a) contacting the sample with one or more lead-binding proteins under conditions sufficient for lead in the sample to bind to the lead-binding protein, wherein said one or more lead-binding proteins comprises a polypeptide selected from the group consisting of acyl-CoA binding protein, thymosin beta-4, and thymosin beta-9, or lead-binding fragments or variants thereof; and
b) detecting the presence of lead in said sample.

11. The method of claim 10, wherein said thymosin beta-4 protein comprises the sequence of SEQ ID NO: 1 or SEQ ID NO: 10.

12. The method of claim 10, wherein said acyl-CoA binding protein comprises the sequence of SEQ ID NO: 2.

13. The method of claim 10, wherein said thymosin beta-9 protein comprises the sequence of SEQ ID NO: 9.

14. The method of claim 10, wherein said one or more lead-binding proteins or lead-binding fragments or variants thereof are labeled with a detectable label, and wherein detection occurs by detecting an alteration of said detectable label.

15. A method for detecting the presence of lead in a sample, comprising:
a) contacting the sample with one or more detectably labeled lead-binding proteins under conditions sufficient for binding of lead to said lead-binding protein, wherein said one or more lead-binding proteins are attached to a solid substrate, and wherein said one or more lead-binding proteins comprises a polypeptide selected from the group consisting of acyl-CoA binding protein, thymosin beta-4, and thymosin beta-9, or lead-binding fragments or variants thereof, and
b) detecting the presence of lead in the sample by detecting said detectable label.

16. The method of claim 15, wherein detecting the presence of lead in said sample comprises contacting said lead-binding protein or lead-binding fragments or variants thereof with a detectable label that differentiates between lead-binding proteins or lead-binding fragments or variants thereof that are bound with lead, and those that are not;
and wherein detection occurs by detecting an alteration of said detectable label.

17. The method of claim 15, wherein said solid substrate comprises a matrix.

18. The method of claim 15, wherein said thymosin beta-4 protein comprises the sequence of SEQ D NO: 1 or SEQ ID NO: 10.

19. The method of claim 15, wherein said acyl-CoA binding protein comprises the sequence of SEQ DD NO: 2.

20. The method of claim 15, wherein said thymosin beta-9 protein comprises the sequence of SEQ ID NO: 9.

21. A method for removing lead from a sample, comprising contacting said sample with one or more lead-binding proteins affixed to a matrix, wherein said one or more lead-binding proteins comprises a polypeptide selected from the group consisting of acyl-CoA binding protein, thymosin beta-4, and thymosin beta-9, or lead-biding fragments or variants thereof; wherein lead in said sample binds to said binding protein, whereby lead is removed from said sample.

22. The method of claim 21, wherein said thymosin beta-4 protein comprises the sequence of SEQ ED NO: 1 or SEQ ID NO: 10.

23. The method of claim 21, wherein said acyl-CoA binding protein comprises the sequence of SEQ ID NO: 2.

24. The method of claim 21, wherein said thymosin beta-9 protein comprises the sequence of SEQ ID NO: 9.

25. A device for detecting the presence of lead in a sample, said device comprising:
a) a solid substrate having attached thereto a matrix;
b) a matrix comprising a ligand, wherein said ligand is diffusable through said matrix and detectable, wherein said ligand specifically binds to lead, and wherein said ligand is localized to a specific area of said matrix; and
c) one or more lead-binding proteins immobilized in said matrix and localized to a specific area of said matrix different from said ligand, wherein said one or more lead-binding proteins comprises a polypeptide selected from the group consisting of acyl-CoA binding protein, thymosin beta-4, and thymosin beta-9, or lead-binding fragments or variants thereof.

26. The device of claim 25, wherein said ligand comprises a chromogen.

27. The device of claim 25, wherein said thymosin beta-4 protein comprises the sequence of SEQ ID NO: 1 or SEQ ID NO: 10.

28. The device of claim 25, wherein said acyl-CoA binding protein comprises the sequence of SEQ ID NO: 2.

29. The device of claim 25, wherein said thymosin beta-9 protein comprises the sequence of SEQ ID NO: 9.

30. The device of claim 25, wherein said area of said matrix containing the ligand and said area of the matrix comprising one or more lead-binding proteins, or lead-binding fragments or variants thereof, are located at opposite ends of said device, separated by matrix that does not contain ligand or protein.

31. A method of detecting the presence of lead in a sample, comprising:
a) contacting the sample with the device of claim 22, wherein the sample contacts the area comprising the ligand under conditions suitable for the lead ill the sample to bind to the ligand;
b) maintaining the device under conditions wherein the lead bound ligand diffuses to the area comprising the lead-binding protein, wherein the lead bound ligand binds to the protein and is thereby immobilized: and
c) detecting the immobilized lead.

32. The method of claim 31, wherein said thymosin beta-4 protein comprises the sequence of SEQ ID NO: 1 or SEQ ID NO: 10.

33. The method of claim 31, wherein said acyl-CoA binding protein comprises the sequence of SEQ ID NO: 2.

34. The method of claim 31, wherein said thymosin beta-9 protein comprises the sequence of SEQ ED NO: 9.

35. The method of claim 31, wherein said ligand comprises a chromogen.

36. A Lit for detection of lead in a sample, said kit comprising the device of claim 25 and one or more reagents for detecting lead in a sample.

37. A kit for removing, lead from a sample, said kit comprising the device of claim 4 and instructions for use of said device.

38. The kit of claim 37, wherein said matrix comprises an absorbent material.


SEQUENCE LISTING (10 sequences)

SEQ ID NO: 1 - protein, 44 aa, Homo sapiens
MSDKPDMAEIEKFDKSKLKKTETQEKNPLPSKETIEQEKQAGES

SEQ ID NO: 2 - protein, 87 aa, Homo sapiens
MSQAEFEKAAEEVRHLKTKPSDEEMLFIYGHYKQATVGDINTERPGMLDFTGKAKWDAWNELKGTSKEDAMKAYINKVEELKKKYGI

SEQ ID NO: 3 - DNA, 64 bp, Homo sapiens
tatgtcagataaaccggatatggctgaaattgaaaaatttgataaatctaaactgaaaaaaacc

SEQ ID NO: 4 - DNA, 72 bp, Homo sapiens
cctgggtttcggtttttttcagtttagatttatcaaatttttcaatttcagccatatccggtttatctgaca

SEQ ID NO: 5 - DNA, 73 bp, Homo sapiens
gaaacccaggaaaaaaacccgctgccgtcaaaagaaaccattgaacaggaaaaacaggctggcgaatcgtaac

SEQ ID NO: 6 - DNA, 67 bp, Homo sapiens
tcgagttacgattcgccagcctgtttttcctgttcaatggtttcttttgacggcagcgggttttttt

SEQ ID NO: 7 - DNA, 37 bp, Artificial Sequence
  synthetic primer used to amplify ACBP gene
gggaattccatatgtctcaggctgagtttgagaaagc

SEQ ID NO: 8 - DNA, 44 bp, Homo sapiens
  synthetic primer used to amplify ACBP gene
taccgctcgagtcaaatcccgtattttttcttcagctcttctac

SEQ ID NO: 9 - protein, 41 aa, Homo sapiens
ADKPDLGEINSFDKAKLKKTETQEKNTLPTKETIEQEKQAK

SEQ ID NO: 10 - protein, 15 aa, Homo sapiens
ETQEKNTLPTKETIE